(19) United States
(12) Patent Application Publication
(10) Pub. No.: US 2013/0197401 A1
(43) Pub. Date: Aug. 1, 2013

(54) OPTIMIZATION OF ULTRASOUND WAVEFORM CHARACTERISTICS FOR TRANSCRANIAL ULTRASOUND NEUROMODULATION

(71) Applicants: Tomo Sato, Roanoke, VA (US); William J. Tyler, Roanoke, VA (US); Daniel Z. Wetmore, San Francisco, CA (US)

(72) Inventors: Tomo Sato, Roanoke, VA (US); William J. Tyler, Roanoke, VA (US); Daniel Z. Wetmore, San Francisco, CA (US)

(21) Appl. No.: 13/731,101

(22) Filed: Dec. 30, 2012

Related U.S. Application Data
(60) Provisional application No. 61/581,905, filed on Dec. 30, 2011.

(51) Int. Cl. A61N 7/00

(52) U.S. Cl. CPC A61N 7/00; A61N 2007/0026
USPC 601/2

(57) ABSTRACT

The present invention relates to methods and systems for achieving effective neuromodulation by transcranial ultrasound (bioTU). Embodiments of the invention include methods and systems for selecting, generating, and delivering transcranial ultrasound to the brain of a living subject. Methods and systems are described for determining the effect of bioTU on brain function. Certain embodiments of the present invention include methods and systems for measuring at least one quantifiable metric of brain activity, cognitive function, or physiology in order to optimize the ultrasound waveforms delivered. In an embodiment, the invention uses a closed-loop design to iteratively improve the effectiveness of bioTU waveforms delivered.

### CROSS-REFERENCE TO RELATED APPLICATIONS

This application claims the benefit of U.S. Provisional Patent Application No. 61/581,905 filed Dec. 30, 2012, the entire disclosure of which is incorporated herein by reference.

### FIELD OF THE INVENTION

The present invention generally relates to methods and systems for achieving effective neuromodulation by transcranial ultrasound (bioTU). Embodiments of the invention include methods and systems for selecting, generating, and delivering transcranial ultrasound to the brain of a living subject. Methods and systems are described for determining the effect of bioTU on brain function. Certain embodiments of the present invention include methods and systems for measuring at least one quantifiable metric of brain activity, cognitive function, or physiology in order to optimize the ultrasound waveforms delivered. In an embodiment, the invention uses a closed-loop design to iteratively improve the effectiveness of bioTU waveforms delivered.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND OF THE INVENTION

Ultrasound (US) has been used for many medical applications, and is generally known as cyclic sound pressure with a frequency greater than the upper limit of human hearing. The production of ultrasound is used in many different fields, typically to penetrate a medium and measure the reflection signature or to supply focused energy. For example, the reflection signature can reveal details about the inner structure of the medium. A well-known application of this technique is its use in sonography to produce a picture of a fetus in a womb. There are other applications which may provide therapeutic effects, such as lithotripsy for ablation of kidney stones or high-intensity focused ultrasound for thermal ablation of brain tumors. An important benefit of ultrasound therapy is its non-invasive nature. US waveforms can be defined by their acoustic frequency, intensity, waveform duration, and other parameters that vary the timecourse of acoustic waves in a target tissue. US waveforms based on repeated pulses less than about 1 second are generally referred to as pulsed ultrasound and are repeated at a rate equivalent to the pulse repetition frequency. Tone bursts that extend for about 1 second or longer—though, strictly speaking, are also pulses—are often referred to as continuous wave (CW).

Ultrasound can be defined as low or high intensity. Ultrasound imaging generally employs high frequency ultrasound (greater than about 1 MHz). In ultrasound, acoustic intensity is a measure of power per unit of cross sectional area (e.g. mW/cm²) and requires averaging across space and time. The intensity of the acoustic beam can be quantified by several metrics that differ in the method for spatial and temporal averaging. These metrics are defined according to technical standards established by the American Institute for Ultrasound in Medicine and National Electronics Manufacturers Administration (NEMA. Acoustic Output Measurement Standard For Diagnostic Ultrasound Equipment (National Electrical Manufacturers Association, 2004)). A commonly used intensity index is the ‘spatial-peak, temporal-average’ intensity (Iₛₚₜₐ). The intensities reported herein refer to Iₛₚₜₐ at the targeted brain region.

Acoustic frequencies greater than about 1 MHz used in ultrasound imaging and most previous ultrasound neuromodulation studies have disadvantages in regard to tissue heating and transmission of mechanical energy. Damage due to ultrasound can occur due to thermal effects (heating) or mechanical effects (such as inertial cavitation—the creation of air bubbles that expand and contract with the time-varying pressure waves). High-intensity US can readily produce mechanical and/or thermal tissue damage, precluding it from use in non-invasive brain-circuit stimulation. High-intensity US (e.g. >1 W/cm²) influences neuronal excitability by producing thermal effects. High-intensity US can readily produce mechanical and/or thermal tissue damage, precluding it from regular use in non-invasive brain-circuit stimulation. These studies delivered ultrasound directly to the brain or periphery. Transcranial delivery of high frequency ultrasound great than about 1 MHz can lead to tissue heating, particularly of bone in the skull. Low-frequency US can be more efficiently transmitted through skull bone, so transcranial US using acoustic frequencies below about 1 MHz can be safely used at higher powers and/or for longer transcranial stimulation protocols.

One important piece of evidence indicating that the mechanism of bioTU is primarily mechanical rather than thermal is that the timecourse of neuromodulation correlates more strongly with the timecourse of mechanical energy transmission than with the timecourse of thermal effects in the tissue. It has been shown that electrophysiological responses to bioTU in mice occur within tens to hundreds of milliseconds of the onset of the bioTU protocol. In contrast, tissue heating occurs on a timescale of 100 s of milliseconds to seconds (Tufail et al., 2010). Moreover, effective bioTU brain stimulation occurred in these mice without tissue heating. In these studies, a 0.87 mm diameter thermocouple (TA-29, Warner Instruments, LLC, Hamden, Conn., USA) was inserted into motor cortex through a cranial window and no deviation in brain temperature greater than the noise level of these recordings (about 0.01 degrees Celsius) was observed (Tufail et al., 2010).

The mechanical effects of US induce neuromodulation before mechanical energy becomes absorbed to a degree such that sufficient tissue heating can occur to affect neural circuit function by thermal means. The acoustic pressure wave begins to affect the mechanosensitivity of lipid bilayers, protein channels, and neuronal membranes at the speed of sound in tissue (microseconds to tens of microseconds). The temporally lagging tissue heating incurred by US tends to be slower than the mechanical effects requiring tens of milliseconds or longer.

The thermal index (TI) of ultrasound is the ratio of power applied to that which would raise the temperature of tissue by 1 degree Celsius. The TI is an important parameter used to assess the heating of tissue due to absorption of energy from the acoustic waves. Bone absorbs ultrasound to a greater degree than other tissues, so TI values for bone are higher for a given ultrasound waveform relative to other tissues. The skull reflects, diffracts, and absorbs acoustic energy fields during transcranial US transmission. The acoustic impedance mismatches between the skin-skull and skull-brain interfaces present additional challenges for transmitting and focusing US through the skull into the intact brain. The absorption of ultrasound by bone is highly dependent on the acoustic frequency with more absorption at frequencies greater than about 1 MHz. Ultrasound below about 0.7 MHz is transmitted more effectively through bone and thus beneficial for bioTU due to reduced heating of the skull. A second reason that bioTU employs lower acoustic frequencies than used for imaging applications is that the mechanical index of ultrasound scales inversely with the square root of the acoustic frequency. Thus, reducing the acoustic frequency by half (e.g. from 1 MHz to 0.5 MHz) increases the mechanical power transmitted to the target tissue by about 1.4 (the square root of 2).

Neuromodulation of the brain by ultrasound has been shown in animals using transcranial ultrasound for neuromodulation (bioTU). Other transcranial ultrasound based techniques use a combination of parameters, including high intensities (greater than about 1 W/cm²) and/or high acoustic frequencies (greater than about 1 MHz) and/or pulsing and waveform parameters, that disrupt or otherwise affect neuronal cell populations so that they do not function properly and/or heat tissue (bone tissue or soft tissue) so as to damage or ablate tissue. bioTU employs a combination of parameters that transmits mechanical energy through the skull to its target in the brain without causing significant thermal or mechanical damage and induces neuromodulation primarily through mechanical means.

Recent research and disclosures have described the use of bioTU to activate, inhibit, or modulate neuronal activity (Tufail et al., 2010; Tufail et al., 2011; Tyler et al., 2008), the full disclosures of which are incorporated herein by reference. Also see U.S. Pat. No. 7,283,861 and US patent applications 20070299370, 20110092800 titled “Methods for modifying currents in neuronal circuits” by inventor Alexander Bystritsky; patent applications by one or more of the named inventors of this submission: patent application Ser. Nos. 13/003,853 (Publication number: US 2011/0178441 A1) titled “Methods and devices for modulating cellular activity using ultrasound” and PCT/US2010/055527 (Publication number: WO/2011/057028) titled “Devices and methods for modulating brain activity”, and commonly assigned patent application No. 61/550,334, titled “Improvement of Direct Communication”; and US patent applications by inventor David J. Mishelevich: Ser. No. 12/917,236 (Publication number: US 2011/0082326 A1) titled “TREATMENT OF CLINICAL APPLICATIONS WITH NEUROMODULATION”; Ser. No. 12/940,052 (Publication number: US 2011/0112394 A1) titled “NEUROMODULATION OF DEEP-BRAIN TARGETS USING FOCUSED ULTRASOUND”; Ser. No. 12/958,411 (Publication number: US 2011/0130615 A1) titled “MULTI-MODALITY NEUROMODULATION OF BRAIN TARGETS”; Ser. No. 13/007,626 (Publication number: US 2011/0178442 A1) titled “PATIENT FEEDBACK FOR CONTROL OF ULTRASOUND DEEP-BRAIN NEUROMODULATION”; Ser. No. 13/020,016 (Publication number: US 2011/0190668 A1) titled “ULTRASOUND NEUROMODULATION OF THE SPHENOPALATINE GANGLION”; Ser. No. 13/021,785 (Publication number: US 2011/0196267 A1) titled “ULTRASOUND NEUROMODULATION OF THE OCCIPUT”; Ser. No. 13/031,192 (Publication number: US 2011/0208094 A1) titled “ULTRASOUND NEUROMODULATION OF THE RETICULAR ACTIVATING SYSTEM”; Ser. No. 13/035,962 (Publication number: US 2011/0213200 A1) titled “ORGASMATRON VIA DEEP-BRAIN NEUROMODULATION”; and Ser. No. 13/098,473 (Publication number: US 2011/0270138) titled “Ultrasound Macro Pulse And Micro Pulse Shapes For Neuromodulation”, the full disclosures of which are incorporated herein by reference). The actual mechanisms underlying bioTU have not been fully elucidated.

An appropriate ultrasound stimulation protocol must be delivered in order to induce changes in the brain via bioTU. The temporal pattern of ultrasound vibration delivered to the brain affects the induced neuromodulation. The temporal pattern of ultrasound waveforms may also affect the nature of the induced neuromodulatory effect such as neuromodulation (which may be mediated by a change in the excitability of neuronal circuits), stimulation of neuronal activity, or inhibition of neuronal activity.

Varying ultrasound waveforms can determine the neuromodulatory effect, if any, of bioTU, but it should be understood that the specific ultrasound waveform parameters that are effective for one use may not be effective in other species, brain targets, ultrasound transducers, or bioTU hardware.

For bioTU, identifying effective or optimal stimulation parameters can be slow and challenging due to the large number of modifiable variables used to define a temporal pattern of ultrasound stimulation. The richness of this parameter space is a beneficial aspect of bioTU that permits ultrasound waveforms to be chosen to generate a desired form of neuromodulation appropriate for the species, brain target, ultrasound transducers, and bioTU hardware. Complex waveforms may be required to achieve particular bio-effects.

Due to the immense parameter space of potential ultrasound waveforms, methods and systems to select efficacious waveforms would be beneficial. Moreover, methods and systems to generate waveforms with advantageous waveform components would also facilitate the practice of effective bioTU neuromodulation.

The major advantages of bioTU for brain stimulation are that it offers a mesoscopic spatial resolution of a few millimeters and the ability to penetrate beyond the brain surface while remaining completely non-invasive. bioTU has beneficial advantages over other forms of non-invasive neuromodulation that include focusing, targeting tissues at depth, and painless stimulation procedures. Ultrasound also offers a rich degree of flexibility for modifying the stimulation protocol. One potentially advantageous aspect of the large parameter space available for bioTU is the possibility of improving the specificity of the induced neuromodulation effect with regard to cell type, sub-cellular compartment, receptor type, or brain structure by varying bioTU parameters. In contrast, other non-invasive forms of brain stimulation are more limited in the extent to which stimulation parameters can be varied. For instance, the spatial extent of TMS is fixed for a given electromagnet. For tDCS, only the location and type of electrodes, current amplitude, and stimulus duration can be varied. Due to its rich parameter space for being able to generate a wide variety of distinct stimulus waveforms yielding different effects on neural activity patterns, bioTU is well-suited for non-invasive brain stimulation.

Methods or systems for generating arbitrary complex ultrasound waveforms for transcranial ultrasound neuromodulation (e.g. bioTU) have not been previously described. Methods and systems that facilitate the generation, selection, and delivery of arbitrarily complex bioTU waveforms would be advantageous.

Therefore, there is a need in the art for systems and methods for generating waveforms of arbitrary complexity for transcranial ultrasound neuromodulation. These and other features and advantages of the present invention will be explained and will become obvious to one skilled in the art through the summary of the invention that follows.

### SUMMARY OF THE INVENTION

Embodiments of the present invention provide systems and methods for identifying effective ultrasound stimulation waveforms for inducing neuromodulation in the brain of a living subject via transcranial ultrasound neuromodulation (referred to herein as bioTU). Embodiments provide systems and methods for selecting, generating, and/or delivering bioTU stimulation protocols, as well as methods and systems for evaluating whether the desired effect was achieved in the subject. Embodiments may incorporate hardware and software components for generating ultrasound protocols (i.e., “stimulation components”). In an embodiment, the invention contains one or a plurality of component devices and systems to measure changes in brain activity, physiology, or cognitive function induced by bioTU to evaluate the efficacy of the bioTU protocol delivered. These measurements provide feedback to improve the selection of subsequent bioTU waveforms. In an embodiment, the invention incorporates algorithms for automatically generating ultrasound stimulation waveforms. In an embodiment, systems and methods are described for storage in an electronic data medium of transcranial ultrasound stimulation parameters (a ‘waveform bank’), the efficacy of the stimulation, and other relevant data so as to improve the algorithms for selecting advantageous ultrasound stimulation parameters. By selecting an appropriate set of ultrasound waveforms and delivering them sequentially to the subject while monitoring changes in brain activity, physiology, or cognitive function, effective bioTU protocols can be efficiently identified.

### BRIEF DESCRIPTION OF DRAWINGS

FIG. 1: bioTU delivery framework.

FIG. 2: System to select, generate, deliver, and assess a set of bioTU waveforms.

FIG. 3: Decision workflow for determining whether to continue searching for efficacious bioTU waveforms.

FIG. 4: bioTU waveform, pulsed ultrasound protocol.

FIG. 5: bioTU waveform, continuous wave ultrasound protocol.

FIG. 6: bioTU waveform repetition.

FIG. 7: bioTU waveform generated by convolution of a delta function and a bioTU waveform component.

FIG. 8: bioTU waveforms with constant or variable pulse repetition frequency.

FIG. 9: bioTU waveforms with variable pulse repetition frequency.

FIG. 10: An example of an amplitude modulated ultrasound waveform.

FIG. 11: Another example of an amplitude modulated ultrasound waveform.

FIG. 12: An example of a sine-wave modulated ultrasound waveform.

FIG. 13: Differential effects of US waveforms on neuronal activity as a function of frequency and intensity (adapted from Tufail et al., 2010).

FIG. 14. An example system for delivering and assessing bioTU protocols.

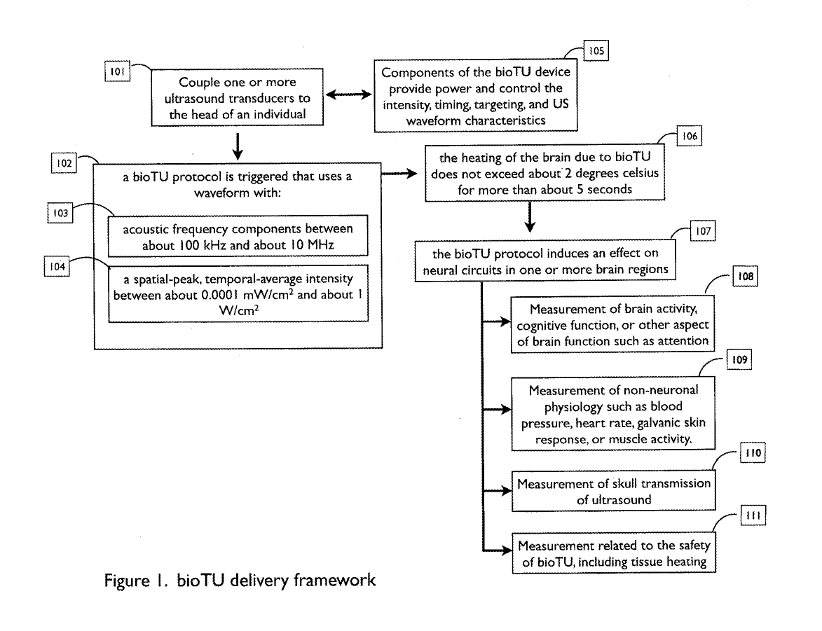

### DETAILED DESCRIPTION

According to an embodiment of the present invention, the methods and systems described herein are related to generating ultrasound waveforms of bioTU protocols. In an embodiment of the invention, one or more components of the invention are used to evaluate the efficacy of a bioTU waveform delivered to a subject by measuring one or more physiological effects, one or more cognitive effects, safety, skull transmission, or other measurements that relate to the safety or efficacy of a bioTU protocol. In some embodiments of the invention, the selection of bioTU protocols is improved over time by recording the resulting neuromodulation—if any—from previous studies, experiments, use cases, and bioTU waveform searches in a relational database. In some embodiments of the invention, bioTU waveforms and bioTU waveform components are also stored in the relational database (also referred to herein as a ‘waveform bank’).

bioTU is a beneficial new technique for modulating brain circuit activity via patterned, local vibration of brain tissue using US having an acoustic frequency greater than about 100 kHz and less than about 10 MHz. In common embodiments, ultrasound energy in a bioTU waveform is present at a range of acoustic frequencies in this range. bioTU transmits mechanical energy through the skull to its target in the brain without causing significant thermal or mechanical damage and induces neuromodulation. bioTU employs low intensity ultrasound such that the spatial-peak, temporal-average intensity (Lₛₚₜₐ) of the bioTU protocol is less than about 1 W/cm² in the targeted brain tissue. The acoustic intensity measure Lₛₚₜₐ is calculated according to established techniques well known to those skilled in the art that relate to the ultrasound acoustic pressure and other bioTU protocol characteristics such as the temporal average power during the bioTU waveform duration. US may be delivered as short-lived continuous waves less than about 5 seconds, in a pulsed manner, or in the form of an ultrasound waveform of arbitrary complexity during bioTU protocols such that diverse patterns of neuromodulation can be induced. For modulating the activity of brain circuits through localized tissue vibration, bioTU protocols may utilize US waveforms of any type known in the art. These include amplitude modulated waveforms, tone-bursts, pulsed waveforms, continuous waveforms, and other waveform patterns that will be described in detail below.

In a preferred embodiment of this invention, bioTU is used to induce neuromodulation in a subject through the use of one or more ultrasound transducers and one or more power and control components. In this preferred embodiment, the one or more ultrasound transducers are coupled to the head of an individual human or animal (the ‘subject’, ‘user’, or ‘recipient’) (101) and the one or more components of the bioTU device are near or wearably attached to the recipient in order to provide power and control the intensity, timing, targeting, and waveform characteristics of the transmitted acoustic waves (105).

In accordance with the above referenced preferred embodiment, the one or more ultrasound transducers and one or more power and control components work in conjunction to trigger a bioTU protocol that uses a waveform that (102) (a) has an acoustic frequency between about 100 kHz and about 10 MHz (103), (b) has a spatial-peak, temporal-average intensity between about 0.0001 mW/cm² and about 1 W/cm² (104), and (c) does not induce heating of the brain due to bioTU that exceeds about 2 degrees Celsius for more than about 5 seconds (106). Further, the bioTU protocol induces an effect on neural circuits in one or more brain regions (107); a ‘bioTU assessment’ quantifies this effect of bioTU on brain function by measuring one or more of the following (108): (a) a subjectively measured response by the recipient as a perception, movement, concept, instruction, other symbolic communication, or by modifying the recipient's cognitive, emotional, physiological, attentional, or other cognitive state (108); (b) an assessment of cognitive function such as a test of motor control, a test of cognitive state, a test of cognitive ability, a sensory processing task, an event related potential assessment, a reaction time task, a motor coordination task, a language assessment, a test of attention, a test of emotional state, a standardized cognitive task, or a customized cognitive task (108); (c) a measurement of brain activity such as electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, or other techniques for measuring brain activity known to one skilled in the art (108); (d) a physiological measurement of the body such as by electromyogram (EMG), galvanic skin response (GSR), heart rate, blood pressure, respiration rate, pulse oximetry, pupil dilation, eye movement, gaze direction, or other physiological measurement known to one skilled in the art (109); (e) a measurement of skull transmission of the delivered ultrasound waveform (110); (f) a measurement related to the safety of bioTU such as thermal effects on the skin, skull, dura, and/or brain; thermal effects on one or more components of the bioTU system; or (g) other safety measurements (111). One of ordinary skill in the art would appreciate that there are numerous methods for providing a quantitative bioTU assessment, and embodiments of the present invention are contemplated for use with any method for providing a quantitative bioTU assessment. In an embodiment of the invention, bioTU is delivered to a subject without providing a quantitative bioTU assessment.

bioTU assessments may be made by one or more bioTU assessment components configured to allow for the measurement of the aforementioned one or more quantifiable effects. bioTU assessment components, include, but are not limited to: psychophysical sensory assessments such as threshold for auditory or visual perception; survey, test, or clinical assessment to probe emotion, cognitive function, or mood; a reaction time test of motor function; a Stroop Test or other assessment of executive function; an assessment of working memory such as an n-back test; an assessment of long-term memory such as a visuospatial memory test; brain recording through surface electroencephalography or another system for noninvasive or invasive brain recording; electrodes placed on muscles and configured with amplifiers and filters to record electromyogram; a pair of electrodes configured to assess skin conductance by passing small current pulses to quantify galvanic skin response; an optical pulse sensor for pulse oximetry or photoplethysmography; a chest-strap heart rate monitor; eye tracking to determine gaze position; and a video based system to quantify pupil dilation. With respect to subjective assessments, embodiments of the present invention may be comprised of one or more user interface components for allowing subjects or other users to input data into the system, or a component thereof, regarding the subject's subjective assessment of the effectiveness of one or more bioTU waveforms. User interface components may include, but are not limited to, keyboards, pointer devices, touchscreens, audio input devices, video input devices, ocular tracking devices, motion tracking devices, or any combination thereof. One of ordinary skill in the art would appreciate that there are numerous types of bioTU assessment components and user interface components that could be utilized with embodiments of the present invention, and embodiments of the present invention are contemplated for use with any type of bioTU assessment components and user interface components.

bioTU employs an ultrasound acoustic waveform that transmits mechanical energy through the skull to its target in the brain without causing damage. bioTU is an advantageous form of brain stimulation due to its non-invasiveness, safety, focusing characteristics, and the capacity to vary bioTU waveform protocols for specificity of neuromodulation.

In some embodiments, bioTU brain stimulation protocols modulate neuronal activity primarily through mechanical means.

The parameters of bioTU are critical for ensuring that neuromodulation occurs without damage. bioTU parameters, described in more detail below, include the use of low intensity (less than about 1 W/cm² at the target tissue), low acoustic frequency (between about 100 kHz and about 10 MHz), and an appropriate pulse repetition frequency, pulse length, waveform duration, and other waveform parameters such that the temperature of the target brain region does not rise by more than about 2 degrees Celsius for a period longer than about 5 seconds. In some specific embodiments, a single pulse is delivered that may be referred to as a continuous wave (CW) pulse by one skilled in the art and extends in time for about longer than 10 ms, about longer than 100 ms, about longer than 1 second, or any length of time up to and including 5 seconds. In embodiments of the invention, one or more power and control components work in conjunction with one or more ultrasound transducers to trigger bioTU waveforms generated by hybridization, convolution, addition, subtraction, phase shifting, concatenation, and/or joining with an overlap for a portion of each of the waveforms for two or more bioTU waveforms or bioTU waveform components. In embodiments of the invention, one or more power and control components work in conjunction with one or more ultrasound transducers to trigger bioTU waveforms for which modulation or ramping of the intensity of all or a portion of the waveform occurs. In embodiments of the invention, one or more power and control components work in conjunction with one or more ultrasound transducers to trigger bioTU waveforms for which modulation or ramping of any other parameter used to define an ultrasound waveform other than intensity occurs.

Appropriate bioTU protocols are advantageous for mitigating or eliminating tissue damage while simultaneously modulating neuronal activity primarily through mechanical means. For example, low temporal average intensity can be achieved by reducing the acoustic power of the ultrasound waves or by varying one or more bioTU parameters to decrease the effective duty cycle—the proportion of time during a bioTU waveform that ultrasound is delivered. Reduced duty cycles can be achieved by decreasing one or more bioTU parameters chosen from pulse length, cycles per pulse, pulse repetition frequency, or other waveform parameters. Low temporal average intensity can be achieved by varying one or more ultrasound parameters during a bioTU protocol. For instance, the acoustic power may be decreased during a portion of a bioTU protocol. Alternatively, the pulse repetition frequency can be decreased during a bioTU protocol. In other embodiments, ultrasound waveforms can be generated that are effective for inducing neuromodulation and maintain an appropriately low temporal average intensity.

Depending on the bioTU protocol, activation or inhibition of brain activity can be achieved. In embodiments of the invention, alternate bioTU stimulation protocols can be chosen in order to specifically activate one or more types of membrane bound, cytoskeletal, or cytoplasmic proteins including ion channels, ion pumps, or secondary messenger receptors. In an embodiment, it is possible to selectively activate or inhibit specific cell types based on their expression of the targeted protein.

A bioTU protocol delivers ultrasound to one or more brain regions and induces neuromodulation that correlates more strongly in time with the timecourse of mechanical effects on tissue than thermal effects. The dominant acoustic frequency for bioTU is generally greater than about 100 kHz and less than about 10 MHz. In common embodiments of bioTU, a mix of acoustic frequencies are transmitted. Particularly advantageous acoustic frequencies are between about 0.3 MHz and 0.7 MHz. The spatial-peak temporal-average (Iₛₚₜₐ) intensity of the ultrasound wave in brain tissue is greater than about 0.0001 mW/cm² and less than about 1 W/cm². Particularly advantageous Lₛₚₜₐ values are between about 100 mW/cm² and about 700 mW/cm².

In many embodiments, the lower limit of the spatial-peak temporal-average intensity (Lₛₚₜₐ) of the ultrasound energy at a target tissue site is chosen from the group of: 21 mW/cm², mW/cm², 30 mW/cm², 40 mW/cm², or 50 mW/cm², for example. In an embodiment of the invention, the upper limit of the Iₛₚₜₐ of the ultrasound energy at a target tissue site is chosen from the group of: 1000 mW/cm², 500 mW/cm², 300 mW/cm², 200 mW/cm², 100 mW/cm², 75 mW/cm², or 50 mW/cm², for example. The Iₛₚₜₐ value for any particular bioTU protocol is calculated according to methods well known in the art that relate to the ultrasound pressure and temporal average of the bioTU waveform over its duration. Effective ultrasound intensities for bioTU do not cause tissue heating greater than about 2 degrees Celsius for a period longer than about 5 seconds.

Significant attenuation of ultrasound intensity occurs at the boundaries between skin, skull, dura, and brain due to impedance mismatches, absorption, and reflection so the required ultrasound intensity delivered to the skin or skull may exceed the intensity at the targeted brain region by up to 10-fold or more depending on skull thickness and other tissue and anatomical properties between the location of an ultrasound transducer and a targeted brain region.

In an embodiment of the invention, providing a mixture of ultrasound frequencies is useful for efficient brain stimulation. Various strategies for achieving a mixture of ultrasound frequencies to the brain of the user are known. Driving an ultrasound transducer at a frequency other than the resonant frequency of the transducer is one way to create ultrasound waves that contain power in a range of frequencies. For instance, an ultrasound transducer with a center frequency of 0.5 MHz can be driven with a sine wave at 0.35 MHz. A second strategy for producing ultrasound waves that contain power in a range of frequencies is to use square waves to drive the transducer. A third strategy for generating a mixture of ultrasound frequencies is to choose transducers that have different center frequencies and drive each at their resonant frequency. A fourth strategy for generating a mixture of ultrasound frequencies is to drive an ultrasound transducer with a waveform that itself contains multiple frequency components. One or more of the above strategies or alternative strategies known to those skilled in the art for generating US waves with a mixture of frequencies would also be beneficial, and embodiments of the present invention are contemplated for use with any strategy for generating US waves with a mixture of frequencies.

Mixing, amplitude modulation, or other strategies for generating more complex bioTU waveforms can be beneficial for driving distinct brain wave activity patterns or to bias the power, phase, or spatial extent of brain oscillations such as slow-wave, delta, beta, theta, gamma, or alpha rhythms.

The effect of bioTU on brain activity may be increased or decreased by the action of at least one of the ultrasound waves, which may include increasing or decreasing one or more of: neuron firing; glial function or trafficking; neurotransmitter receptor receptivity; release or uptake of neurohormones, neurotransmitters or neuromodulators; gene transcription; protein translation; protein phosphorylation; cell trafficking of proteins or mRNA; and metabolic activity of a brain cell.

In some embodiments, bioTU can be delivered from a phased array of transducers for improved targeting of one or more brain regions. Constructive and destructive interference of acoustic waves transmitted by multiple transducers can be used to deliver complex spatiotemporal patterns of acoustic waves. Moreover, the spectral density of acoustic pressure profiles delivered to a targeted brain region can be varied to produce differential effects on neuronal activity. These properties of bioTU offer the possibility of activating widely distributed brain networks. In certain embodiments, the capacity to target distributed brain regions concurrently or with a specific order further extends the possibilities for modulating brain activity. In an alternative embodiment, a plurality of ultrasound transducers are employed for delivering bioTU to a subject and the bioTU waveform delivered from some or all ultrasound transducers differs in one or a plurality of parameters that may include intensity, acoustic frequency, pulse duration, pulse repetition frequency, or another parameter that defines the bioTU waveform.

A device for brain stimulation using bioTU includes a single component or a plurality of components to generate, transduce, and couple ultrasound acoustic waves to the head of a human or animal. A power source provides power to the various components of the device including one or more of function generators, controllers, radio frequency (RF) power amplifiers, ultrasound transducers, or any combination thereof. In certain embodiments, a computer or other controller hardware with general or custom software is used to control the timing and protocol parameters of bioTU. In alternate embodiments, control of the timing and protocol parameters can be accomplished through the use of one or more digital or analog components, operating with or without the inclusion of any general or custom software.

In one embodiment, a first function generator (FG1) is used to trigger US pulses, establish the pulse-repetition frequency (PRF) and define the number of pulses (np) in a bioTU stimulus waveform. FG1 triggers a second function generator 2 (FG2) that establishes the acoustic frequency (Af) and the number of cycles per pulse (cpp) in a bioTU stimulus waveform. An RF amplifier receives a voltage waveform input from FG2 and provides output power to an ultrasound transducer that generates the acoustic wave of the bioTU stimulus. Systems and methods, including hardware and software, for generating ultrasound waveforms of arbitrary complexity will be described in greater detail below.

Various ultrasound transducers can be used to generate the acoustic wave. Specific water immersion type transducers are the Ultran GS500-D13, NDT Systems IBMF0.53, Ultran GS350-D19, Olympus Panametrics V318 focused transducer 0.5 MHz/0.75″ F=0.85″, Ultran GS200-D25 and Olympus Panametrics V301S 0.5 MHz/1.0″. Customized ultrasound transducers designed with appropriate intensity and resonant acoustic frequency characteristics may also be advantageous for delivering bioTU. For instance, a Blatek AT21926 Rev 0 transducer tuned to 300 kHz may be beneficial for bioTU.

For the vast majority of transducers (air-coupled transducers being an exception), the ultrasound device must be in physical contact with the subject due to the poor impedance match between air and tissue. Ultrasound gel (or another coupling material) is usually used to couple the transducer apparatus to the head to minimize distortion or reflection of the ultrasound waveform due to acoustic impedance mismatch. In some embodiments of a bioTU device, components for cooling are used due to heating that can occur in the transducer, coupling gel, brain, and/or body. Although some components of the bioTU device may be placed remotely from the subject, transducers other than air-coupled transducers require physical attachment to the subject in this embodiment. The subject's head may be placed in an assembly that holds the transducer assembly in contact with the user. Alternatively, the transducer apparatus may be wearably attached to the user with a helmet, headband, adhesive material, hat, eyeglasses, or other piece of wearable hardware or clothing.

bioTU can be delivered in a targeted manner to activate a specific brain region. Alternatively, a bioTU device can be unfocused in order to modulate the activity of multiple brain regions, a cerebral hemisphere, or other large areas up to the size of the entire brain.

Several strategies are known for targeting bioTU to a specific brain region. When using water-matched transducers, the transmission of US from the transducer into the brain only occurs at points at which acoustic gel (or other coupling fluid) physically couples the transducer to the head. On the basis of this acoustic transmission property, coupling the transducer to the head through small gel contact points represents one physical method for transmitting US into restricted brain regions. In this embodiment, the entire face of the transducer should always be covered with acoustic gel to prevent heating and damage of the transducer face. The area of gel coupling the transducer to the head, however, can be sculpted to restrict the lateral extent through which US is transmitted into the brain. Although this method does provide an effective approach for stimulating coarsely targeted brain regions, calculating acoustic intensities transmitted into the brain with this method can be difficult because of nonlinear variations in the acoustic pressure fields generated.

Alternatively, the lateral extent of the spatial envelope of US transmitted into the brain can be restricted by using acoustic collimators. Single-element transducers having concave focusing lenses or transducers shaped to deliver a targeted acoustic wave can also be used for delivering focused acoustic pressure fields to brains. Such single-element focused transducers can be manufactured having various focal lengths depending on the lens curvature, as well as the physical size and center frequency of the transducer. The most accurate yet complicated US focusing method involves the use of multiple transducers operating in a phased array.

Beneficial embodiments target one or more brain regions chosen from the group of brain regions that: mediate sensory experience, motor performance, and the formation of ideas and thoughts, as well as states of mood, emotion, physiological arousal, sexual arousal, attention, creativity, relaxation, empathy, connectedness, and other cognitive states. In some embodiments, bioTU is targeted to modulate neuronal activity underlying multiple sensory domains and/or cognitive states concurrently or in close temporal arrangement.

The capacity for targeting any brain region non-invasively is one beneficial aspect of bioTU. Due to the effective transmission of ultrasound waves through tissue, bioTU permits neuromodulation throughout the brain. Distinct brain regions are known to mediate specific cognitive functions. Other aspects of brain function are highly distributed. One or more brain regions may be targeted concurrently to achieve the desired neuromodulatory effect for the user.

In some embodiments of the invention, ultrasound waves for bioTU are targeted to areas of the cerebral cortex. The cerebral cortex is composed of four lobes: the frontal, parietal, occipital, and temporal lobes. The frontal lobe underlies motor planning, motor control, executive control, decision-making, pain-processing, social cognition, and many other higher cognitive functions. Sub-regions of frontal cortex have been identified that underlie these and other specific processes. The parietal lobe is involved in sensory processing, some aspects of motor control such as gaze control, and a variety of other functions. The occipital lobe is primarily involved in visually processing. The temporal lobe mediates auditory processing, many aspects of language production and reception, and important aspects of long-term memory. Various regions of cerebral cortex are sensory processing areas, including: striate visual cortex, visual association cortex, primary and secondary auditory cortex, somatosensory cortex, primary motor cortex, supplementary motor cortex, premotor cortex, the frontal eye fields, prefrontal cortex, orbitofrontal cortex, dorsolateral prefrontal cortex, ventrolateral prefrontal cortex, and anterior cingulate cortex. bioTU targeted to one or more of the above listed regions of cerebral cortex can modulate related cognitive processes or motor commands by activating, inhibiting, or otherwise modulating the function of neuronal circuits.

In other embodiments of bioTU, deeper brain regions are targeted. A non-exhaustive list of brain regions that may be targeted includes: the limbic system (including the amygdala), hippocampus, parahippocampal formation, entorhinal cortex, subiculum, thalamus, hypothalamus, white matter tracts, brainstem nuclei, cerebellum, or other brain region. An alternative embodiment employs a strategy of targeting brain regions underlying the function of a neuromodulatory system.

Some forms of bioTU can be achieved without targeting a specific brain region. For instance, diffuse regions of cerebral cortex have been shown to be sensitive to reward. Moreover, brain oscillations such as slow-wave, delta, beta, theta, gamma, or alpha rhythms are created by the synchronous activation of populations of neurons that may be distributed in non-contiguous brain regions. bioTU protocols designed to oscillate at frequencies consistent with a brain rhythm of interest can be targeted broadly to one or more brain regions known to mediate that form of brain oscillation. For instance, slow-wave oscillations occur in a concerted manner in regions of cerebral cortex that may be discrete or extend through an entire hemisphere. Another embodiment of bioTU to affect brain rhythms could modulate thalamocortical oscillations by targeting the thalamus, sharp-wave ripples by targeting the CA3 region of the hippocampus, or alpha waves by modulating 8-12 Hz rhythms that originate in the occipital lobe. In alternative embodiments, other brain rhythms or distributed neuronal pathways are targeted by bioTU. For each of the targeted rhythms, bioTU may be used in some embodiments to enhance the rhythms and in other embodiments to reduce the rhythms.

At the instructed time, a bioTU protocol is delivered to stimulate the targeted region of the brain in order to activate, inhibit, or modulate its activity and induce an altered subjective experience or cognitive state for the user. Specific embodiments of neuromodulation are described herein and include stimulation targeting primary sensory cortex, primary and secondary motor cortex, association cortex (including areas involved in emotion, executive control, language, and memory), neuromodulatory pathways, the amygdala, the hippocampal formation, and other brain regions. In an embodiment of the invention, the bioTU protocol affects one or more of the attentional state, emotional state, or cognitive state of the recipient. In alternative embodiments of the invention, the bioTU protocol is configured to cause one or more of the following effects: the user is induced to consciously or unconsciously perform an act; the user experiences a state of physiological or sexual arousal; or the user perceives a sensory stimulus.

The temporal pattern of ultrasound vibration delivered to the brain affects the induced neuromodulation. The temporal pattern of ultrasound waveforms may also affect the nature of the induced neuromodulatory effect such as neuromodulation (which may be mediated by a change in the excitability of neuronal circuits), stimulation of neuronal activity, inhibition of neuronal activity, or modulation of one or a plurality of the following biophysical or biochemical processes: (i) ion channel activity, (ii) ion transporter activity, (iii) secretion of signaling molecules, (iv) proliferation of the cells, (v) differentiation of the cells, (vi) protein transcription of cells, (vii) protein translation of cells, (viii) protein phosphorylation of the cells, or (ix) protein structures in the cells. In some embodiments, bioTU may induce different effects concurrently in different brain regions. In some embodiments, bioTU may induce effects in non-targeted brain regions.

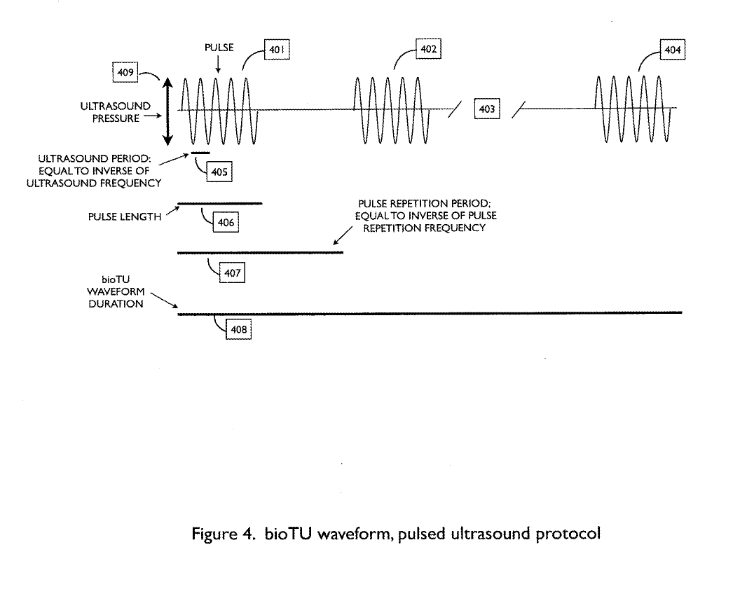

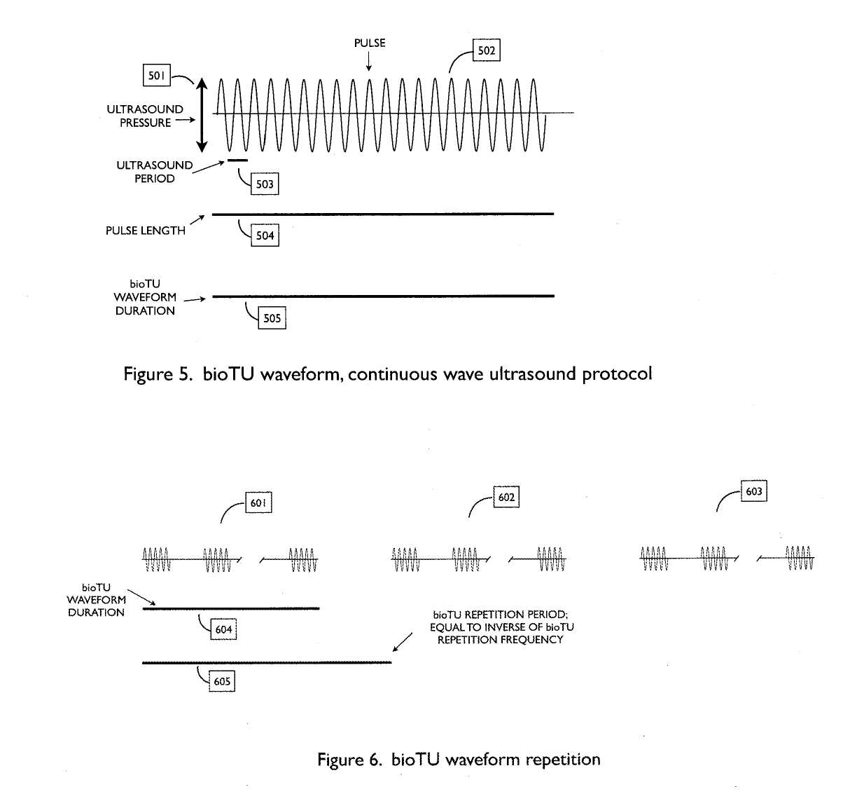

Pulsing of ultrasound is an effective strategy for activating neurons that reduces the temporal average intensity while also achieving desired brain stimulation or neuromodulation effects. In addition to acoustic frequency (405) and transducer variables, several waveform characteristics such as cycles per pulse, pulse repetition frequency, number of pulses, and pulse length affect the intensity characteristics and outcome of any particular bioTU stimulus on brain activity. A pulsed bioTU protocol generally uses pulse lengths (406) between about 0.5 microseconds and about 1 second. A bioTU protocol may use pulse repetition frequencies (PRFs) between about 50 Hz and about 25 kHz (407). Particularly advantageous PRFs are generally between about 1 kHz and about 3 kHz. For pulsed bioTU waveforms, the number of cycles per pulse (cpp) is between about 5 and about 10,000,000. Particularly advantageous cpp values vary depending on the choice of other bioTU parameters and are generally between about 10 and about 250. In FIG. 4, the 1st (401), 2nd (402), and nth (404) pulses are shown, with the gap in the horizontal line (403) indicating additional pulses. In this embodiment, the number of pulses defines the bioTU waveform duration (408). In some embodiments, particularly advantageous pulse numbers for pulsed bioTU waveforms are between about 100 pulses and about 250 pulses. In alternative embodiments, a higher number of pulses is delivered up to about 500,000 pulses.

Tone bursts that extend for about 1 second or longer—though, strictly speaking, also pulses—are often referred to as continuous wave (CW). In alternative embodiments, one or more continuous wave (CW) ultrasound waveforms less than about five seconds in duration (501, 502, 503, 504, 505) is directed to the brain to induce neuromodulation. US protocols that include such CW waveforms offer advantages for neuromodulation due to their capacity to drive activity robustly. However, one disadvantage of bioTU protocols with CW pulses is that the temporal average intensity is significantly higher which may cause painful thermal stimuli on the scalp or skull and may also induce heating and thus damage in brain tissue. Thus, advantageous embodiments using CW pulses may employ a lower acoustic intensity and/or a slow pulse repetition frequency of less than about 1 Hz. For instance, a CW US stimulus waveform with 1 second pulse lengths repeated at 0.5 Hz would deliver US every other second. In alternative embodiments of the invention, pulsing protocols include those with slower pulse repetition frequencies of less than about 0.5 Hz or less than about 0.1 Hz or less than about 0.01 Hz or less than about 0.001 Hz. In some useful embodiments, the interval between pulses or pulse length may be varied during a bioTU protocol.

In some embodiments, repeating the bioTU protocol is advantageous for achieving particular forms of neuromodulation. In some embodiments, the number of times a bioTU protocol of appropriate duration (604) is repeated is chosen to be in the range between 2 times and 100,000 times. FIG. 6 (601, 602, 603) presents a schematic of three repeated bioTU protocols. Particularly advantageous numbers of bioTU protocol repeats are between 2 and 1,000 repeats. In an embodiment of the invention, the bioTU repetition frequency (605) of a bioTU protocol is chosen to be less than about 10 Hz, less than about 1 Hz, less than about 0.1 Hz, or lower. In an embodiment of the invention, the bioTU repetition frequency is configured to be fixed or variable. In embodiments of the invention, variable bioTU repetition frequency values are modulated randomly, pseudo-randomly, according to a linear or non-linear ramped pattern, or otherwise modulated. The bioTU repetition period is defined as the inverse of the bioTU repetition frequency.

Effective and ineffective parameters for ultrasound neuromodulation have been described previously (e.g. (Tufail et al., 2010; Tyler et al., 2008), patent application Ser. Nos. 13/003,853 (Publication number: US 2011/0178441 A1) titled “Methods and devices for modulating cellular activity using ultrasound” and PCT/US2010/055527 (Publication number: WO/2011/057028) titled “Devices and methods for modulating brain activity” by inventor Tyler).

A comprehensive theoretical understanding of the relationship between ultrasound waveform parameters and bioTU efficacy has not been achieved.

The temporal pattern of ultrasound vibration delivered to the brain affects the induced neuromodulation. In order to increase the utility of transcranial ultrasound for neuromodulation (bioTU), new systems and methods are required for selecting, generating, delivering, and determining the efficacy of bioTU ultrasound waveforms of arbitrary complexity. The devices and methods described herein permit potentially efficacious ultrasound waveforms to be selected and delivered via ultrasound transducers to the brain of a subject. In an embodiment of the invention, neuromodulatory efficacy is determined by one or more of: appropriate physiological monitoring of the brain or body, cognitive testing, and self-reporting by the subject. In an embodiment of the invention, selection of ultrasound waveforms is improved by incorporating previous insight about efficacious and/or non-efficacious waveforms stored in a database that may optionally include metadata about the user, brain target, bioTU system, and other information.

The bioTU system and related methods described herein are based on a foundation of a subset of effective waveform components and insights about the physiological effect of bioTU based on previous experimental studies. Various bioTU protocols are delivered iteratively while monitoring the brain and/or body for desired physiological responses and determining the effectiveness of each bioTU protocol. Computational and/or statistical algorithms are used to select the bioTU protocols delivered in order to explore multi-dimensional parameter space efficiently.

Herein we describe systems and methods for delivering bioTU to a subject, with embodiments comprising one or more of: 1) one or a plurality of automatic computerized or manually operated components to select an ultrasound waveform to deliver to a subject; 2) one or a plurality of components for generating a bioTU ultrasound waveform in a form that can be used to drive an ultrasound transducer; 3) one or a plurality of components for delivering a bioTU protocol to a subject, including one or a plurality of ultrasound transducers functionally coupled to the scalp for transmitting ultrasound waves into the brain of the subject; 4) one or a plurality of components for quantifying one or a plurality of: (i) the effect of a bioTU protocol on neuronal function; (ii) the effect of a bioTU protocol on brain activity; (iii) the effect of a bioTU protocol on cognitive function; (iv) the effect of a bioTU protocol on another physiological processes; (v) the safety profile of the bioTU protocol; or (vi) the amount of acoustic energy transmitted into the brain; and 5) methods and systems for selecting different bioTU waveforms to subsequently deliver to a subject such that the selected bioTU waveform is selected with the goal of optimizing one or a plurality of the measurements listed above in (4).

In some embodiments of the invention, the system may include a database (or other data store) in a computer-readable medium (a ‘waveform bank’) for storing bioTU waveforms. In some embodiments of the invention, the waveform bank also stores metadata, such as the specifications about the bioTU system used, and data about the subject who received a bioTU protocol, including, but not limited to, number and type of waveforms previously performed on the subject.

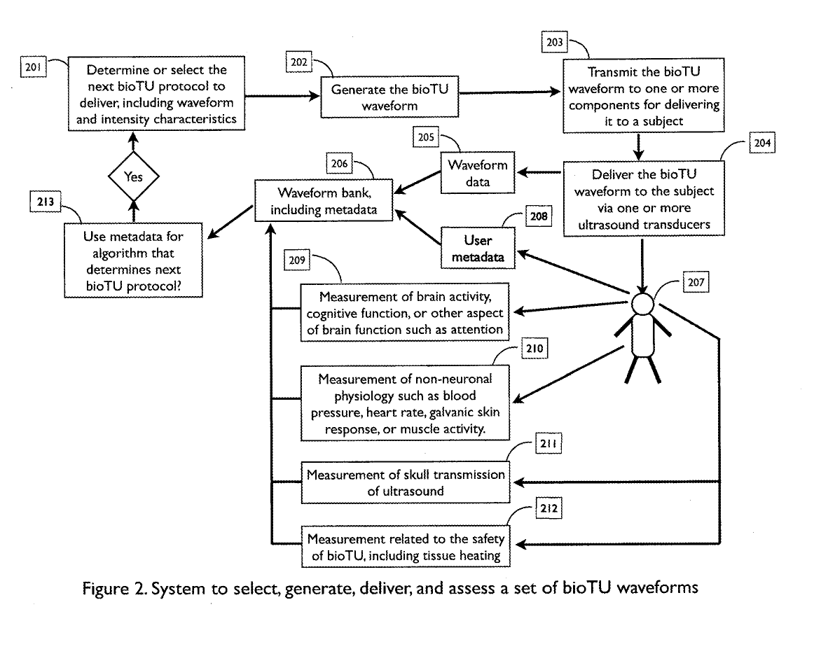

A schematic description of one embodiment of the invention is shown in FIG. 2. Two important features of the embodiment shown in FIG. 2 are (1) a closed loop design and (2) sequential delivery of distinct bioTU waveforms. This embodiment of the present invention incorporates a closed-loop design in which at least about 10 bioTU waveforms are sequentially delivered to the subject and the effect of the bioTU waveforms delivered are assessed and compared. A first bioTU waveform is automatically or manually selected or a bioTU waveform is derived algorithmically by using one or more mathematical equations (201). In some embodiments of the invention, a ‘waveform bank’ (206) is accessed as part of the system or method for selecting or generating a bioTU waveform (213). In some embodiments of the invention, metadata stored in the waveform bank is used for selecting or generating a bioTU waveform.

Hardware and/or software components of the system generate the selected ultrasound waveform (202), then transmit the specified waveform (203) to one or more ultrasound transducers functionally coupled to the head of a subject (207) to deliver the ultrasound waveform (204).

One or more ‘bioTU assessments’ are made to quantify the effect of the bioTU protocol on the subject (209, 210, 211, 212). The at least one ‘bioTU assessment’ measures one or more of safety (212), efficacy as measured by a recording of brain activity (209), cognitive function (209), other physiological measurement (210), and/or efficiency of ultrasound transmission to the targeted brain region (211). The results of the ‘bioTU assessment’ are stored in a ‘waveform bank’ (206) locally by a component of the device or transmitted via a local area network (LAN) or wide area network (WAN) (e.g., the Internet) for storage on a remote computing device (e.g., server) or other remote storage device (e.g., backup drive, flash storage, network accessible storage device). In some embodiments of the invention, the waveform bank stores data about the bioTU waveform (205). In some embodiments of the invention, the waveform bank stores user metadata (208). In some embodiments of the invention, the metadata (and/or other data stored in the waveform bank) is used algorithmically to determine the next bioTU protocol to deliver (213). A second bioTU waveform is automatically or manually selected from a ‘waveform bank’ (201) or derived algorithmically by using one or more mathematical equations. Hardware and/or software components of the system generate the second selected ultrasound waveform (202, 203, 204), and a second ‘bioTU assessment’ is made (209, 210, 211, 212). According to a preferred embodiment of the invention as described herein, a minimum of 10 bioTU waveforms are assessed by the system and methods herein described, including the steps of selecting, generating, delivering, and assessing bioTU waveforms. Certain embodiments may allow for fewer than 10 bioTU waveforms to be assessed. Similarly, certain embodiments of the present invention may allow for additional or fewer steps for use in delivering and assessing bioTU waveforms.

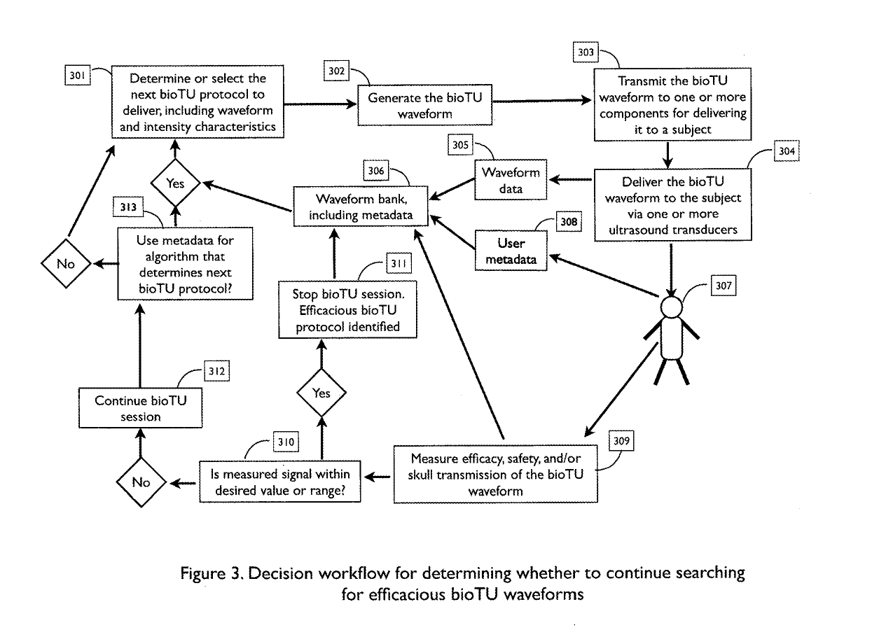

In some embodiments of the invention, the at least one measurement about the safety, efficacy, or skull transmission (309) of the bioTU waveform for a user (307) is compared to a target or threshold value to determine whether an additional bioTU waveform will be selected (301), generated (302), transmitted to a device wearably attached to a user (303), and delivered to the subject (304). In some embodiments of the invention, data concerning the bioTU waveform (305) and user metadata (308) are stored in a waveform bank (306). The at least one parameter measured by a ‘bioTU assessment’ is compared to previous iterations of the system (310). If at least 10 bioTU waveforms have been assessed and the measured signal is within a desired range or has reached a threshold value, the bioTU session is stopped due to the identification of an appropriately efficacious bioTU protocol (311). Certain embodiments may allow for fewer than 10 bioTU waveforms to be assessed before stopping the bioTU session according to the embodiment shown in FIG. 3. Certain embodiments may allow for greater than 10 bioTU waveforms to be assessed before stopping the bioTU session according to the embodiment shown in FIG. 3. If the measured signal is not within a desired range and has not reached a threshold value, the bioTU session continues (312). In some embodiments, metadata stored in the waveform bank contributes to the determination of the subsequent bioTU protocol (313). In some embodiments of the invention the measured effect of bioTU (309) is compared to previous values by comparing to data stored in the waveform bank (306).

In some embodiments of the invention, the ‘bioTU assessment’ is compared to a threshold value, reference value, or other desired value to determine whether continued iterations of selecting, generating, delivering, and assessing bioTU waveforms are required. Continued bioTU protocols are delivered to the subject until either (1) an appropriately effective and safe bioTU protocol is identified or (2) a maximum number of bioTU protocols or maximum time of the bioTU session is reached. This process is repeated to deliver additional bioTU waveforms in order to improve the efficacy or safety profile of the bioTU protocol.

In embodiments of the invention, the process of selecting, generating, delivering, and assessing for safety, efficacy, or both safety and efficacy, is repeated more than about 10 times, about more than 15 times, about more than 20 times, about more than 25 times, about more than 30 times, about more than 35 times, about more than 40 times, about more than 45 times, about more than 50 times, about more than 75 times, about more than 100 times, about more than 200 times, about more than 250 times, about more than 300 times, about more than 400 times, about more than 500 times, about more than 1000 times, or about more than 10000 times. In other embodiments of the present invention, the process may be repeated in fewer or greater iterations than the numbers outlined above and further the process may include additional or fewer steps than outlined above.

Embodiments of the invention in which the repeated process of selecting, generating, delivering, and assessing the effect of bioTU waveforms occurs quickly are beneficial. In alternative embodiments of the invention, the sweep through multiple bioTU waveforms occurs in less than about 1 second, less than about 2 seconds, less than about 3 seconds, less than about 4 seconds, less than about 5 seconds, less than about 10 seconds, less than about 20 seconds, less than about 30 seconds, less than about 40 seconds, less than about 50 seconds, less than about 1 minute, less than about 2 minutes, less than about 3 minutes, less than about 4 minutes, less than about 5 minutes, less than about 6 minutes, less than about 7 minutes, less than about 8 minutes, less than about 9 minutes, less than about 10 minutes, less than about 20 minutes, less than about 30 minutes, less than about 40 minutes, less than about 50 minutes, or less than about 1 hour.

In various embodiments of the invention, the time between delivering bioTU protocols to a subject is less than about 10 minutes, less than about 5 minutes, less than about 4 minutes, less than about 3 minutes, less than about 2 minutes, less than about 1 minute, less than about 50 seconds, less than about 40 seconds, less than about 30 seconds, less than about 20 seconds, less than about 10 seconds, less than about 5 seconds, less than about 4 seconds, less than about 3 seconds, less than about 2 seconds, less than about 1 second, less than about 500 milliseconds, less than about 250 milliseconds, less than about 100 milliseconds, less than about 50 milliseconds, less than about 25 milliseconds, less than about 10 milliseconds, less than about 5 milliseconds, less than about 4 milliseconds, than about 3 milliseconds, than about 2 milliseconds, or less than about 1 millisecond. In some embodiments of the invention, the time between delivering bioTU protocols to a subject is fixed. In alternative embodiments of the invention, time between delivering bioTU protocols to a subject is variable. In embodiments of the invention with variable intervals between bioTU protocols, the intervals are random, pseudo-random, or structured according to another irregular pattern.

According to an embodiment of the present invention, the system incorporates hardware and software components for generating ultrasound protocols of arbitrary complexity. Complex waveforms can be generated by any technique known in the art for generating control signals for driving one or a plurality of ultrasound transducers and related components. In most embodiments, voltage-varying waveforms will be generated by dedicated software and/or hardware.

In some embodiments of the invention, ultrasound waveforms are generated algorithmically using one or a plurality of mathematical equations. In some embodiments, combinatorial techniques are used to generate bioTU waveforms. In alternative embodiments, bioTU waveforms are generated by adding, subtracting, hybridizing, concatenating, convolving, or otherwise combining two or more bioTU waveforms or bioTU waveform components. In common embodiments, bioTU waveforms take the form of pulse trains of ultrasound. According to these various embodiments, pulse trains are generated by adding, subtracting, hybridizing, concatenating, convolving, or otherwise combining two or more bioTU pulse trains. Triggering is an effective and simple strategy for generating a variety of bioTU waveforms. In some embodiments, multiplying and dividing bioTU waveforms or bioTU waveform components is used to generate complex bioTU waveforms. In alternative embodiments of the invention, multiple bioTU waveforms or bioTU waveform components are combined with temporal offsets and/or voltage offsets. In yet other embodiments, a combination of more than one method for generating bioTU waveforms is used, such as a combination of triggering and adding, subtracting, hybridizing, concatenating, convolving, or otherwise combining two or more bioTU waveforms. For instance, a bioTU waveform can be generated by triggering a particular bioTU waveform or bioTU waveform component upon the occurrence of a threshold crossing event of another slower sinusoidal waveform.

According to an embodiment of the present invention, an ultrasound pulse is generated by brief bursts of square waves, sine waves, saw-tooth waveforms, sweeping waveforms, or arbitrary waveforms, or combinations of one or more waveforms. In some embodiments the ultrasound energy transmitted according to the waveforms is focused. In alternative embodiments of the invention, the ultrasound energy transmitted according to the waveforms is not focused. The method may be repeated or applied in single applications. The components for generating ultrasound, such as an ultrasound transducer or its elements, are driven using analog and/or digitized waveforms. Ultrasound transducer elements may be driven using individual waveforms or a combination of waveforms from the group of waveforms including, but not limited to, square, sine, saw-tooth, arbitrary waveforms or any combination thereof. In an embodiment of the invention, ultrasound pulses for bioTU are sine waves having a single ultrasound frequency. In an embodiment of the invention, ultrasound pulses for bioTU are composed of oscillating shapes other than sine waves, such as square waves, or spikes, or ramps, or a pulse that includes multiple ultrasound frequencies composed of beat frequencies, harmonics, or a combination of frequencies generated by constructive or deconstructive interference techniques, or some or all of the aforementioned. Individual pulses can be shaped by superimposing pulse trains on the base ultrasound carrier and heterogeneous patterns of pulse shaping with sine waves, square waves, triangular waves, or arbitrarily shaped waves.

Although many distinct ultrasound waveforms can be generated by previous disclosed techniques, the ultrasound waveforms taught by the prior art are few in number relative to those that can be created by the systems and methods described herein. In short, prior art teaches ultrasound waveforms for bioTU that are few in number. In contrast, an infinite number of ultrasound waveforms are possible according to the methods and systems described herein for generating more complex waveforms.

For instance, programmable function generators can be used for manually generating bioTU waveforms. Integrating multiple programmable function generators allows more complex waveforms to be generated. For instance, a first function generator can be programmed to transmit a 5V control signal that represents the period of the entire bioTU waveform. A 300 millisecond bioTU protocol delivered to a subject every 30 seconds would require a 300 msec 5V signal followed by 27.7 seconds at 0V for an effective duty cycle of 1%. In this example, the first function generator is connected to the input of a second function generator that creates pulses. For instance, a bioTU waveform with a pulse repetition frequency of 1 kHz and pulse duration of 100 μl is output by FG2.

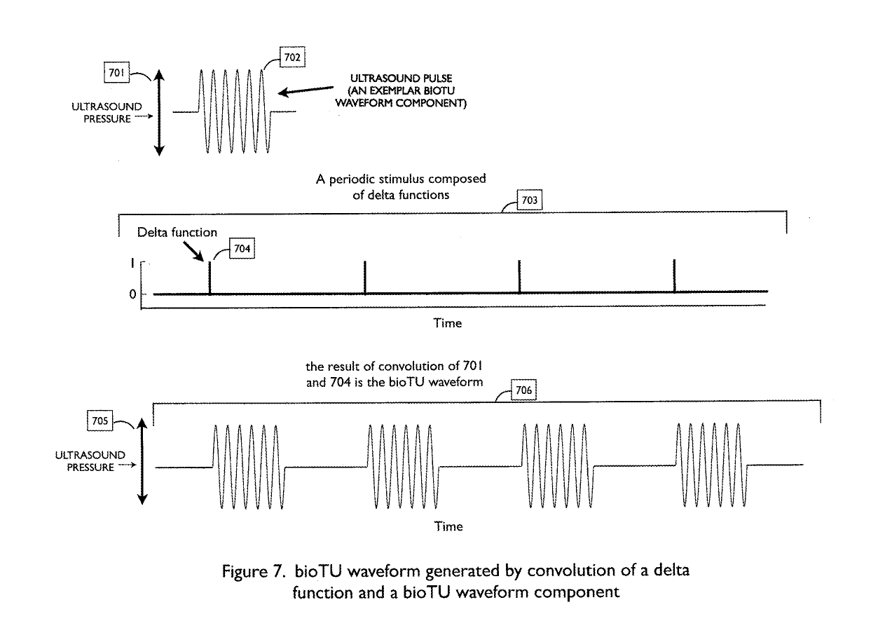

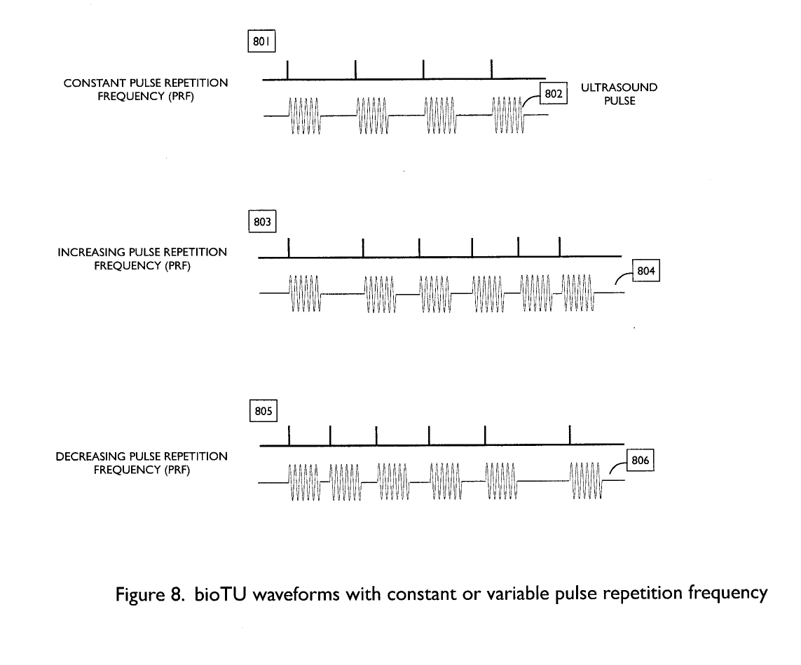

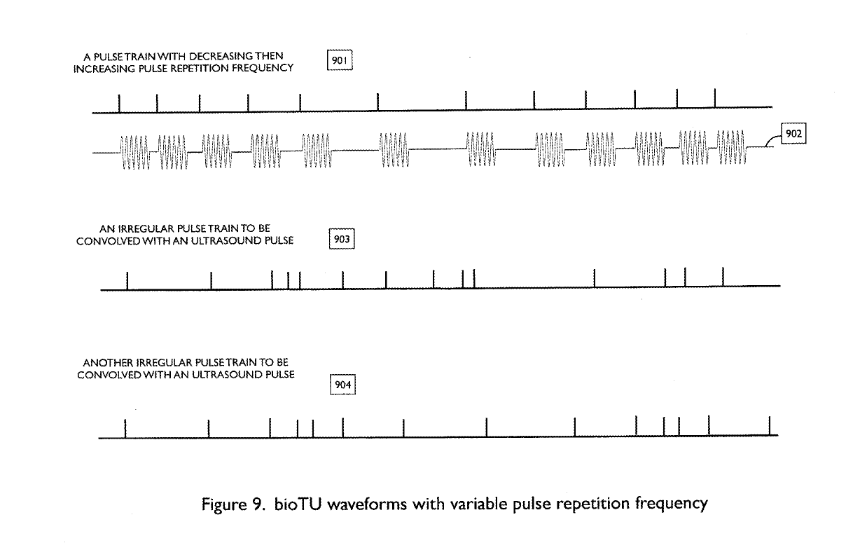

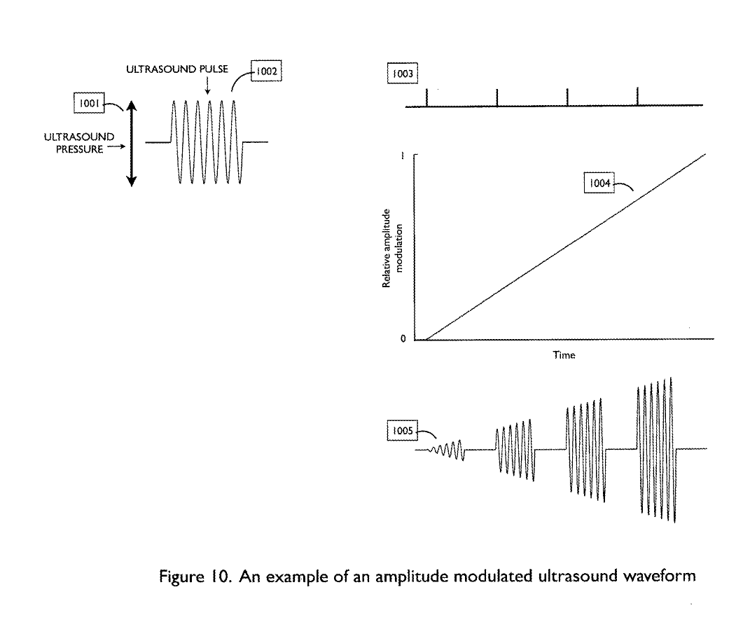

An example of a bioTU waveform generated by convolving a delta function and a bioTU waveform component is shown in FIG. 7. A bioTU waveform component—a pulse of ultrasound (702) defined by acoustic frequency and the ultrasound pressure (701)—is convolved with a pulse train (703) defined mathematically as a set of delta functions (704). The resulting ultrasound waveform is a regular train of ultrasound pulses (705, 706). Additional exemplar pulse trains are shown in FIG. 8 for pulsed ultrasound waveforms defined based on a regular pulse repetition frequency (801, 802), an increasing pulse repetition frequency (803, 804), and a decreasing pulse repetition frequency (805, 806). Further exemplar pulse trains are shown in FIG. 9: a pulse train with a decreasing then an increasing pulse repetition frequency (901, 902), and two irregular sequences of delta functions to be convolved with an ultrasound pulse (903, 904).

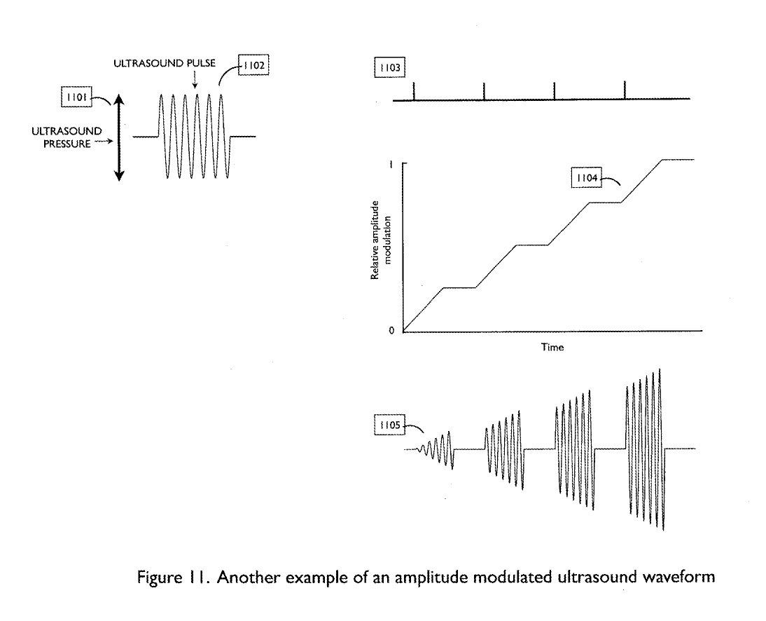

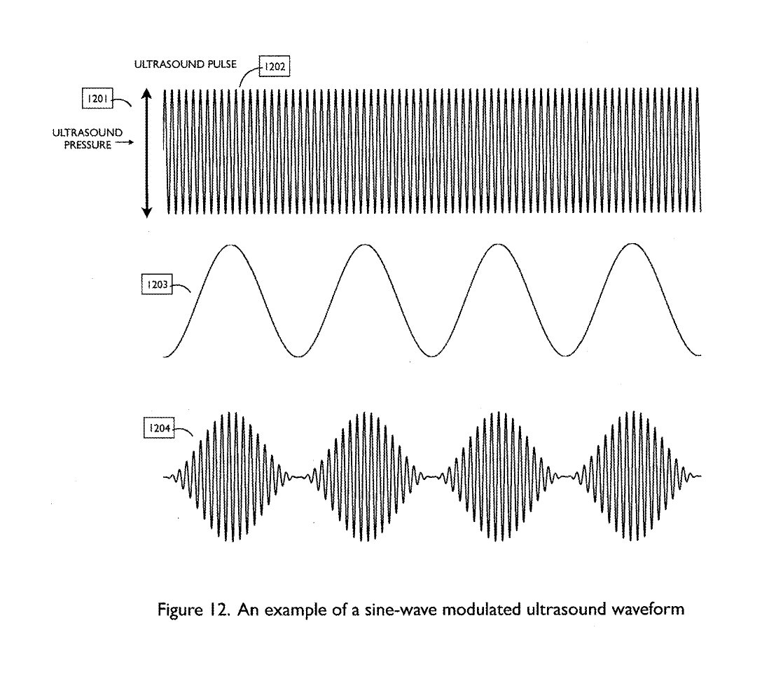

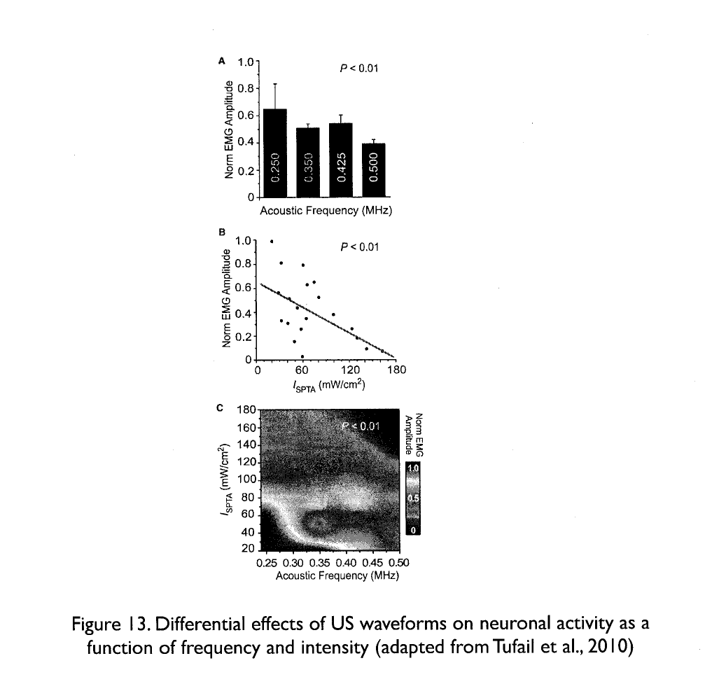

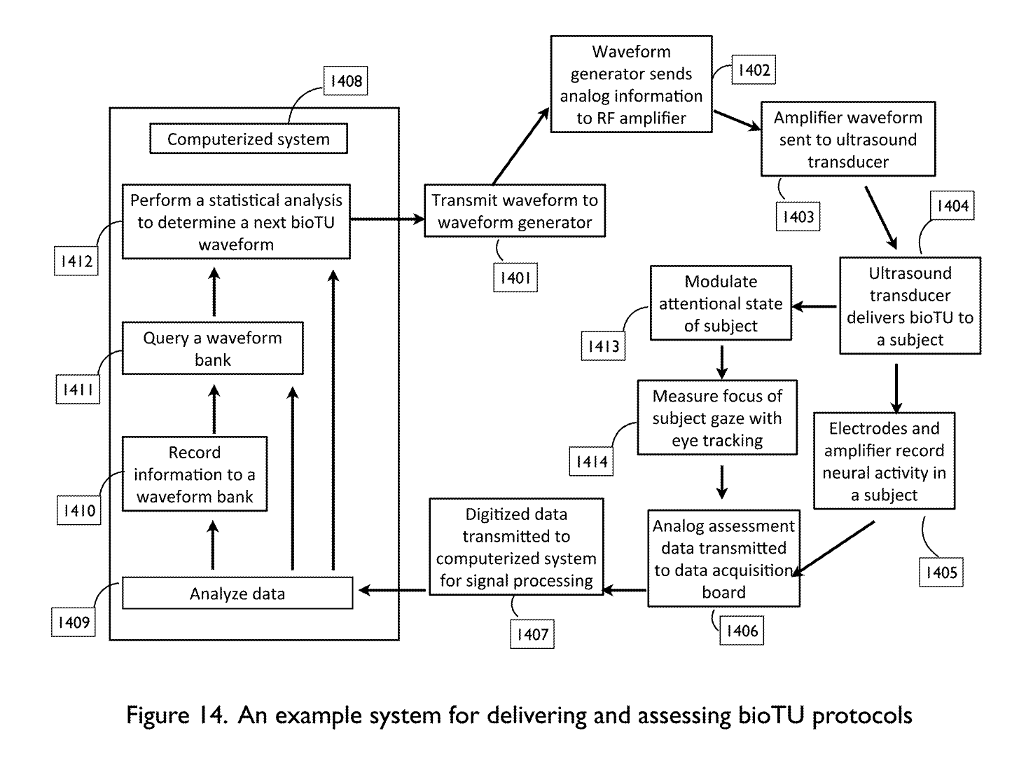

Amplitude modulation of one or more bioTU waveform parameters is a beneficial strategy for generating complex bioTU waveforms. In an embodiment of the invention shown in FIG. 10, an ultrasound pulse (1001, 1002) is convolved with a sequence of delta functions (1003) and modulated according to a linear ramp (1004) to generate an amplitude modulated pulse train bioTU waveform component (1005). In an alternative embodiment of the invention shown in FIG. 11, an ultrasound pulse (1101, 1102) is convolved with a sequence of delta functions (1103) and modulated according to a different linear ramp that does not modulate the amplitude of the bioTU waveform between pulses (1104) to generate an alternative amplitude modulated pulse train bioTU waveform component (1105). In yet another alternative embodiment of the invention shown in FIG. 12, a longer ultrasound pulse (1201, 1202) is modulated by a sine wave function (1203) to generate a sine wave amplitude modulated bioTU waveform component (1204).

It should be understood that the examples described herein and shown in FIGS. 7, 8, 9, 10, 11, and 12 are a brief portion of a bioTU waveform that would in many embodiments extend longer in time. It should also be understood that the waveforms described herein and plotted in figures are a small subset of bioTU waveforms that can be generated and delivered according to the systems and methods described herein.

More complex bioTU waveforms can also be generated using one or more programmable function generators. Alternatively, complex waveforms are generated with appropriate software such as Matlab (Mathworks, Natick, Mass.) or LabVIEW (National Instruments, Austin, Tex.), then communicated by electronic components via a wired or wireless communication protocol to one or more components of the system that transduce ultrasound acoustic waves and couple them to the subject transcranially.

The system described herein has the potential to generate an infinite number of bioTU waveforms. The large number of potential bioTU protocols is an advantageous feature of the invention. In some embodiments of the invention, delivering complex ultrasound waveforms is beneficial for achieving the desired neuromodulatory effect of bioTU.

In some embodiments of the invention, complex bioTU waveforms are required to achieve particular bio-effects, changes to cognitive processes, or otherwise induce neuromodulation. A non-exclusive list of the benefits of being able to create a more variable set of bioTU waveforms includes the possibility of: achieving a wider range of physiological effects; reaching brain regions that otherwise cannot be targeted; accounting for individual differences in skull transmission; and optimizing a bioTU waveform to reduce safety concerns such as tissue heating. Although not intending to be restricted to any one theory for the characteristics that determine bioTU efficacy, different bioTU waveforms may be more efficacious depending on variables including one or more of the group: the brand, model, resonant frequency, maximum power output, or other specifications of the one or more ultrasound transducers; the specifications of the at least one function generator, controllers, radio frequency (RF) power amplifiers, computer or other controller hardware, software, or other component of the bioTU device; the location of the one or more brain regions targeted, including the depth of the one or more brain region targeted and structures in the one or more paths to that brain region which may affect the spatial extent, intensity, or acoustic frequencies present at the targeted brain tissue; the specific neuromodulatory effect desired including neuromodulation, neuronal stimulation, and/or neuronal inhibition; the thickness and acoustic properties of skin, scalp, skull, dura, brain tissue, and ventricles underlying the one or more ultrasound transducers; time-of-day; user's sleep stage, cognitive state, emotional state, level of physiological arousal, level of sexual arousal, or other aspect of the user's cognitive function; the user's age, sex, geographic location, medical history, disease state, height, weight, skull thickness, genetic information, diet, other health data, or other behavioral information; and the user's brain activity as measured by electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, or other techniques for measuring brain activity known to one skilled in the art.

According to a preferred embodiment of the present invention, a bioTU system is configured to achieve the desired neuromodulatory effect in a relatively short period of time. Thus, one beneficial aspect of these preferred embodiments of the invention are methods and systems to efficiently sweep multiple bioTU waveforms to identify effective bioTU protocols. In other embodiments, where subjects are available to be subject to the bioTU protocols for longer durations, the system can be configured for more systematic application, allowing for detailed analysis and determination of optimized bioTU waveforms for a particular subject.

Given the high dimensional parameter space and infinite number of potentially efficacious bioTU protocols, it is advantageous to have a system that improves the efficiency of identifying sufficiently or optimally efficacious bioTU protocols. A component of preferred embodiments of the present invention is a relational database, lookup table, data store or other data storage system (waveform bank'). The waveform bank contains information about bioTU waveforms delivered during previous bioTU sessions or available to be used for future bioTU sessions that is a component of, or otherwise capable of communicating with and controlling the one or more ultrasound transducers of a bioTU device. The waveform bank comprises a plurality of, or waveform components of a plurality of, ultrasound stimulation waveforms. The waveform bank is advantageous for storing, selecting, and automatically generating ultrasound waveforms that are effective for the characteristics of a particular bioTU session. Components of the device include one or a plurality of control units configured to select from the waveform bank, or construct from waveform components in the waveform bank, a bioTU waveform or sequence of bioTU waveforms.

The ‘waveform bank’ is used to improve the selection of bioTU protocols based on results from previous studies, experiments, use cases, and bioTU sessions. In some embodiments of the invention, the amount of insight gained from accessing, analyzing, or otherwise using the data stored in the waveform bank increases over time as additional data about bioTU sessions are incorporated into the waveform bank.

In some embodiments of the invention, the waveform bank includes metadata. In beneficial embodiments of the invention, metadata is stored in the waveform bank.

In some embodiments of the invention, the waveform bank includes bioTU protocols for activation of multiple brain regions concurrently or with a specified temporal delay. In some embodiments of the invention, the relational database is dynamic and capable of modification based on feedback from one or more users, manual modification by a skilled practitioner of brain stimulation techniques, or other automated or semi-automated algorithms. In some embodiments of the invention, the relational database exists on a device near or wearably attached to the user, on a device near or wearably attached to the user that includes one or a plurality of devices for brain stimulation, or in a remote location on a server operated by a company, government agency, military force, first responder department, or community group. In some embodiments of the invention, the database also exists in multiple copies at a plurality of locations.

In embodiments of the invention, the waveform bank is stored on electronic media in any form known to one skilled in the art of database design. In some embodiments, the waveform bank is stored in a database system that is a component of a system wearably attached or near to the user. In alternative embodiments, the waveform bank is stored in a database system remote from the user that connects to a bioTU system wearably attached to the user directly by a wireless or wired communication protocol or via the Internet or other local or wide area network. In some embodiments, the waveform bank stores metadata including one or a plurality from the group of: bioTU waveform parameters, hardware components for delivering bioTU, software associated with hardware components for delivering bioTU, the intended brain target, the intended neuromodulatory effect, the intended change to cognitive state, cognitive function, or sensory processing, and metadata about the user's health, genetics, behavior, emotional state, physical characteristics, diet, drug use (approved prescription drugs and illegal drugs), alcohol use, or other characteristic of the user.

In some embodiments of the invention, the waveform bank includes a plurality of, or the waveform components of a plurality of, bioTU waveforms of the waveform bank taken from the group consisting of waveforms generated using analog circuits, digital waveforms or components thereof, including numbers selected from tables or generated by evaluating mathematical functions. In some embodiments of the invention, the waveform bank includes noise signals.

In some embodiments of the invention, the waveform bank is updated after a bioTU waveform has been delivered to a subject. In some embodiments of the invention, the waveform bank describes one or a plurality of parameters of a bioTU waveform. In some embodiments of the invention, the waveform bank is updated after a sub-set of bioTU waveforms is delivered to a subject. In some embodiments of the invention, the waveform bank is a component of the bioTU system wearably attached to the user. In some embodiments of the invention, the waveform bank is stored remotely from the bioTU system wearably attached to the user. In some embodiments of the invention, information is transmitted to or from the waveform bank and the bioTU system wearably attached to the user by a wireless or wired protocol. In some embodiments of the invention, information is transmitted via the Internet, local area network, wide area network or any combination thereof, to or from the waveform bank and the bioTU system wearably attached to the user by a wireless protocol.

In some embodiments of the invention, the waveform bank stores metadata associated with bioTU waveforms or bioTU waveform components. In some embodiments of the invention, the stored metadata includes one or a plurality of data types selected from: (a) subject metadata; (b) bioTU metadata; (c) data concerning the components used to deliver bioTU for the stored event; and/or (d) data about the transmission of ultrasound into the brain through the skin, skull, dura, and/or brain.

In embodiments of the invention, subject metadata includes, but is not limited to one or a plurality of: (i) subject identifying information including one or a plurality of name, address, social security number, email address, login information for a third party service such as Facebook, Google, or Twitter, assigned coded identifier, or other identification information; (ii) age, sex, geographic location, medical history, disease state, height, weight, skull thickness, skull shape, genetic information, diet, other health data, cognitive abilities, cognitive disabilities, or other behavioral information.

In embodiments of the invention, bioTU metadata includes, but is not limited to, one or a plurality of: (i) data concerning the at least one targeted brain region; (ii) data concerning safety of bioTU such as thermal effects of bioTU on hair, scalp, skin, skull, dura, brain tissue, or other tissue; (iii) data about the actual targeting of ultrasound energy as measured by electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, or other techniques for measuring brain activity known to one skilled in the art; (iv) data concerning bioTU efficacy measured by one or a plurality of: (1) subjective experience by the recipient that takes the form of one or a plurality of: a sensory perception, movement, concept, instruction, other symbolic communication, or by modifying the recipient's cognitive, emotional, physiological, attentional, or other cognitive state; (2) measurement of brain activity that takes the form of one or a plurality of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, or other techniques for measuring brain activity known to one skilled in the art; (3) physiological measurement of the body that takes the form of one or a plurality of: electromyogram (EMG), galvanic skin response (GSR), heart rate, blood pressure, respiration rate, pulse oximetry, pupil dilation, eye movement, gaze direction, or other physiological measurement known to one skilled in the art; or (4) a cognitive assessment that takes the form of one or more of: a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, or a customized cognitive task.

In embodiments of the invention, data concerning the components used to deliver bioTU for the stored event include, but are not limited to, one or a plurality of: (i) the number and locations on the head of the at least one ultrasound transducer; (ii) the specifications of the at least one ultrasound transducer; and (iii) the specifications of the at least one function generator, controllers, radio frequency (RF) power amplifiers, computer or other controller hardware, software, or other component of the bioTU device.

In a preferred embodiment of the present invention, the waveform bank uses a computer-readable medium to store the data structure and/or instructions to execute the method. In some embodiments of the invention, the waveform bank is connected to one or more remote servers or other computing devices via a local area network, wide area network, the Internet or any combination thereof. This connection can be beneficially employed for backup purposes, for sharing data between users or between a user and a company, researcher, or other entity, or for improving optimization algorithms by integrating bioTU protocol data and metadata across users. For instance, information from multiple users targeting the same brain region with bioTU protocols can be analyzed together to determine bioTU waveforms that are likely to induce the intended neuromodulatory effect in a particular user. Alternative analytical techniques that incorporate metadata can be used to deliver optimized bioTU protocols based on categorization of users according to demographic, behavioral, neuroanatomical, or other characteristics. For instance, metadata about a user's age, sex, height, weight, skull shape, or skull thickness may affect the transmission of ultrasound waves and be accounted for by delivering an appropriate bioTU waveform. Analysis of demographic segmentation of previous bioTU sessions that included bioTU waveform optimization is beneficial in some embodiments.

In some embodiments of the invention, data saved in the waveform bank includes optimal parameters for a user, bioTU system, brain target, or intended neuromodulatory effect. Stored data can be accessed to determine optimal bioTU parameters for future bioTU sessions by the user. In related embodiments of the invention, the starting point of a bioTU waveform sweep is chosen based on previous optimization (e.g. for a different target) for a user. In some embodiments of the invention, information stored in the waveform bank is used to define one or a plurality of: the first bioTU waveform of a bioTU waveform sweep; the second bioTU waveform of a bioTU waveform sweep; the last bioTU waveform of a bioTU waveform sweep; the n^{th} bioTU waveform of a bioTU waveform sweep where n is greater than two; the sequence of bioTU protocols in a sweep (or sequence) of waveforms delivered during a bioTU session; one or a plurality of bioTU waveforms or bioTU waveforms components included in a bioTU session; one or a plurality of bioTU waveforms or bioTU waveforms components excluded from a bioTU session; or one or a plurality of benchmark bioTU waveforms or bioTU waveform components repeated at least twice during a bioTU session, where a benchmark bioTU waveform is defined according to a known or expected response for a given bioTU protocol as measured by a change in brain activity, physiological measurement, or cognitive state.

In some embodiments of the invention, data stored in a waveform bank from bioTU sessions with other users is used to select one or a more bioTU waveforms in a bioTU session. In one embodiment, one or more bioTU waveforms or bioTU waveform components are chosen based on multiple previous bioTU sessions in subjects other than the current user for which a particular brain target and/or neuromodulatory or cognitive effect previously occurred. In some embodiments of the invention, data from bioTU sessions in other users is used to select one or more bioTU waveforms to include in a bioTU session. In some embodiments of the invention, data from bioTU sessions in other users is used to exclude one or more bioTU waveforms from a bioTU session. In various embodiments of the invention, data stored in the waveform bank that is used for selecting bioTU waveforms comes from more than 2 users, more than 3 users, more than 4 users, more than 5 users, about more than 10 users, about more than 15 users, about more than 25 users, about more than 50 users, about more than 100 users, about more than 1000 users, or about more than 10000 users.

In various embodiments of the invention, efficacious bioTU waveforms are selected or generated using one or more components of the invention by employing one or more of the following techniques: algorithmically by using one or more mathematical equations; by selecting waveforms described in a list or table of values; by selecting a specific bioTU waveform; by selecting one or more bioTU waveform components; or by adjusting one or more parameters that define the bioTU waveform chosen from the group of: one or more acoustic frequencies, pulse length, bioTU waveform duration, cycles per pulse, number of pulses, modulation of pulse shape by a ramp, sine wave, square wave, saw-tooth wave, triangle wave, or arbitrary waveform; modulation of any parameter by a ramp, sine wave, square wave, saw-tooth wave, triangle wave, or arbitrary waveform; or other parameters. Ultrasound parameters can be selected randomly, pseudo-randomly, or generated using statistical techniques for instance according to fuzzy logic.

In some embodiments, bioTU waveforms are selected automatically by one or more computerized components of the bioTU system. In some embodiments, bioTU waveforms are selected manually by the recipient of the bioTU waveform, by a skilled practitioner of bioTU, or by one with less experience than a skilled practitioner of bioTU such as a friend, colleague, or other individual. In embodiments in which a bioTU waveform is selected automatically by one or more computerized components of the bioTU system, an algorithm achieved through software running on a computerized or other digital system or via an appropriately designed analog circuit generates the waveform.

In some embodiments of the invention, a sequence of bioTU waveforms is pre-selected. In some embodiments, a set of bioTU waveforms is pre-selected and the order of their presentation is random, pseudo-random, chaotic, selected statistically for instance according to fuzzy logic, or adjusted dynamically based on responses to bioTU waveforms already presented in the sequence as measured by a change in brain activity, physiological measurement, or cognitive state.

In some embodiments of the invention, metadata stored in a waveform bank or relational database is used to determine the bioTU protocols tested. Similarly, metadata contained in the waveform bank can be used in some embodiments to select the sequence of bioTU protocols tested. In some embodiments, efficacious bioTU protocols are efficiently identified based on information stored in the waveform bank that relate to previous bioTU sessions that share one or more characteristics with the current bioTU session. Shared characteristics may include one or more from the group of: species, individual, health or wellness information about the individual, demographic information about the individual, brain target, transducer location, transducer specifications, intended neuromodulatory effect, or other characteristic relevant to the bioTU session.

In some embodiments of the invention, a set of bioTU waveforms delivered during a bioTU session achieve a sweep of values of a single parameter that defines the ultrasound waveform. A non-exhaustive list of parameters that can be used to define an ultrasound waveform that can be varied during a sweep of a single parameter includes: intensity (also referred to as ultrasound pressure), acoustic frequency, pulse repetition frequency, pulse length, number of pulses, modulation of any ultrasound parameter by a ramp or other function, pulse shaping, and bioTU waveform length.

In alternative embodiments of the invention a set of bioTU waveforms delivered during a bioTU session achieve a multi-dimensional sweep of values of more than one parameter that define the ultrasound waveform. In various embodiments of the invention, the multi-dimensional sweep varies more than one parameter, more than two parameters, more than three parameters, more than four parameters, more than about five parameter, more than about ten parameters, more than about 20 parameters, more than about 30 parameters, more than about 40 parameters, or more than about 50 parameters during the multi-dimensional sweep. In some embodiments of the invention that achieve a multi-dimensional sweep during a bioTU session, a sub-set of parameters is kept constant during a portion of the bioTU session. For instance, one parameter is fixed for the first half of the bioTU session and a different, second parameter is kept fixed during the second half of the bioTU session. In another embodiment, a plurality of parameters is kept fixed for a portion of the bioTU session. In some embodiments, different sets of parameters are kept fixed for various portions of the bioTU sessions. A non-exhaustive list of parameters that can be used to define an ultrasound waveform that can be varied during a multi-dimensional sweep includes: intensity (also referred to as ultrasound pressure), acoustic frequency, pulse repetition frequency, pulse length, number of pulses, modulation of any ultrasound parameter by a ramp or other function, pulse shaping, and bioTU waveform length.

In some embodiments of the invention, some of the repeated bioTU waveforms are identical. Using a benchmark stimulation protocol is a well-known technique in physiology, including brain stimulation, to account for a changing baseline response. Potential mechanisms underlying a changing baseline response include habituation or sensitization of neuronal circuits. In some beneficial embodiments, a repeated bioTU protocol induces a benchmark response for comparison to other bioTU waveforms presented. In one embodiment, a benchmark bioTU protocol is used for every other bioTU protocol presented, every third bioTU protocol presented, every fifth bioTU protocol presented, or less frequently. In some embodiments, the frequency of repeating an identical bioTU protocol is irregular, random, or pseudo-random.

In some embodiments, a plurality of transducers is used wherein each of the ultrasound transducers delivers an identical bioTU protocol. In alternative embodiments, a plurality of transducers delivers identical bioTU waveforms that are phase shifted. In yet another embodiment, a plurality of ultrasound transducers delivers distinct bioTU protocols. In some embodiments, a sub-set of the multiple ultrasound transducers deliver an identical bioTU protocol or phase shifted bioTU protocol, while another subset of ultrasound transducers delivers one or more different bioTU protocols.

One or more components of the system are configured to assess the efficacy of a bioTU waveform on a subject. In some embodiments, the invention contains component devices and systems to measure one or more of changes in: brain activity, physiology, cognitive function, or other changes in the brain or body induced by transcranial ultrasound. The measured response to bioTU is used to provide closed loop feedback to other components of the system so as to improve the selection of subsequent bioTU waveforms. In some embodiments of the invention, data concerning the effect of a bioTU waveform on brain activity, physiology, cognitive function, or other changes in the brain or body are transmitted to the one or more components of the invention that select and/or generate one or more subsequent bioTU waveforms. In some embodiments of the invention, data concerning the effect of a bioTU waveform on brain activity, physiology, cognitive function, or other changes in the brain or body are stored in a waveform bank. In some embodiments of the invention, data concerning the effect of a bioTU waveform on brain activity, physiology, cognitive function, or other changes in the brain or body are used for other bioTU sessions by the same user or a different user.

In some embodiments of the invention, one or a plurality of components are used to assess the efficacy of a bioTU protocol by measuring brain activity, physiology, cognitive function, or other changes in the brain or body induced by bioTU. In various embodiments of the invention, brain activity is measured by one or more techniques chosen from the group of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, or other techniques for measuring brain activity known to one skilled in the art.

In various embodiments of the invention, physiology is measured by one or more techniques chosen from the group of: electromyogram (EMG), galvanic skin response (GSR), heart rate, blood pressure, respiration rate, pulse oximetry, pupil dilation, eye movement, gaze direction, or another physiological measurement. A simple ohmeter is effective for measuring skin conductance for assessing the galvanic skin response. A small current is passed between two leads placed near each other on the skin and the conductance is measured. Blood pressure, body temperature, and heart rate can be measured using a sphygmomanometer, thermometer, and pulse oximeter, respectively. These various measurements can be decoded to determine a cognitive state, sleep state, physiological state, or thought, sensory perception, emotion, concept, or state of physiological arousal, sexual arousal, or attention.

In various embodiments of the invention, cognitive function is assessed by one or more testing techniques chosen from the group of: a test of motor control, a test of cognitive state, a test of cognitive ability, a sensory processing task, an event related potential assessment, a reaction time task, a motor coordination task, a language assessment, a test of attention, a test of emotional state, a standardized cognitive task, or a customized cognitive task.

In various embodiments of the invention, an invasive or noninvasive measurement of one or a plurality of components in the circulating blood stream or cerebrospinal fluid is used to assess the effect of bioTU.

In some embodiments of the invention, continuous or intermittent monitoring of the effect of bioTU occurs. The response to a bioTU waveform is continuously or intermittently monitored by one or a plurality of: recording brain activity, making a physiological measurement, assessing cognitive state or cognitive function, and monitoring the extent of transcranial transmission with one or a plurality of ultrasound transducers or other means for measuring acoustic energy known to one skilled in the art.

In some embodiments of the invention, bioTU is targeted to two brain regions wherein one brain region is the primary targeted brain region and the other, secondary brain region is functionally connected to the primary targeted region such that stimulation of the secondary brain region is used to determine the effectiveness of bioTU targeted to the first region. A similar strategy has been previously employed for targeting deep brain stimulation electrodes as described in U.S. Pat. No. 6,253,109 to inventor Gielen titled “System for optimized brain stimulation”.

In some embodiments, one or more control units is configured to assess the safety of bioTU stimulation. In some embodiments of the invention, safety of a bioTU waveform is an assessment of the thermal effects of bioTU. Temperature measurements can be made by one or more techniques including by use of a thermistor, thermometer, camera-based system (e.g. an infrared camera), or other technique. In various embodiments of the invention, temperature measurements can be made of one or more of: coupling gel or other physical system for coupling ultrasound into the body; ultrasound transducer; other components of the ultrasound system; or hair, skin, skull, dura, or brain. Increased temperature in the brain is known to affect the function of neurons and neural circuits—and thus may affect cognitive state and/or cognitive function. In some embodiments of the invention, thermal effects of bioTU are assessed indirectly by making one or more measurements of brain activity, physiology, cognitive state, or cognitive function.

In some embodiments, one or more components of the system assess the efficiency of transmission of the ultrasound wave through the skin, skull, dura, and/or brain. Feedback is provided concerning the quality of a particular bioTU waveform by assessing the effectiveness of ultrasound transmission to the targeted brain region. For instance, the thickness of the skull, orientation of the skull relative to the at least one ultrasound transducer, and other acoustic properties of the skull are significant determinants of the intensity, distribution of acoustic power at different acoustic frequencies, and spatial extent of a transcranial ultrasound wave in the brain. bioTU waveforms for which a large proportion of the ultrasound intensity is absorbed by the skull are less advantageous for transcranial ultrasound neuromodulation, because they have the potential to cause more heating of the skull than waveforms for which more power is transmitted into the brain. Acoustic frequency is one important determinant of absorption of ultrasound energy by the skull. Acoustic frequencies less than about 1 MHz are advantageous for transmission through the skull. Acoustic frequencies less than about 0.7 MHz are particularly advantageous for transmission through the skull.

In some embodiments of the invention, one or more ultrasound transducers are used to detect the signature of reflected ultrasound as is done commonly in ultrasound imaging. This can be accomplished by using a pulse-echo strategy using ultrasound transducers with a dominant acoustic frequency of more than about 1 MHz. By measuring the relative power of reflected ultrasound with different bioTU waveforms, the amount of ultrasound energy absorbed, reflected, or scattered by the skull can be determined. Ultrasound energy reflected by the skull or other part of the head or brain will return to the transducer for measurement more quickly than ultrasound energy reflected by other structural features in the brain. The timing of the expected reflected ultrasound waves can be calculated using techniques from diagnostic ultrasound imaging that are well-known to those skilled in the art of ultrasound imaging. In this embodiment, bioTU waveforms for which less ultrasound energy is measured by the transducer are more effective for neuromodulation because more energy is being transmitted through the skull.

In another embodiment of the invention, the amount of ultrasound energy transmitted through the skull is measured by one or a plurality of transducers on the opposite side of the skull from the one or plurality of ultrasound transducers used for generating the bioTU waveform. In this embodiment, the transducers used for measuring ultrasound on the contralateral side of the skull measure the amount of ultrasound energy transmitted through the skull. In this embodiment, bioTU waveforms for which more ultrasound energy is measured by the one or plurality of transducers are more effective for neuromodulation because more energy is being transmitted through the skull.

In another embodiment of the invention, one or a plurality of methods for measuring acoustic energy that do not include an ultrasound transducer such as by using a fiber optic hydrophone, photoacoustic imaging or another method for measuring acoustic energy known to one skilled in the art are used to quantify the amount of ultrasound energy transmitted through the skull, skin, dura, and brain tissue or reflected by the skull, skin, dura, and brain tissue. In this embodiment, a similar strategy is used as that discussed above for estimating the amount of ultrasound energy that reaches the targeted region of the brain.

In an embodiment of the invention, ultrasound waveforms for bioTU that are formed by the combination of one or a plurality of bioTU waveforms or one or a plurality of bioTU waveform components are advantageous for neuromodulation. In some embodiments of the invention, novel waveforms are generated by varying stimulation parameters or combining waveform components to generate a hybrid ultrasound stimulation waveform. In various embodiments of the invention, one or more techniques for combining waveforms are chosen from the list of: hybridization, convolution, addition, subtraction, phase shifting, concatenation, joining with an overlap for a portion of each of the waveforms, modulation or ramping of the intensity of all or a portion of the waveform, or modulation or ramping of any other parameter used to define an ultrasound waveform. In alternative embodiments of the invention, bioTU waveforms and the order of their presentation during a bioTU session is generated online algorithmically based on pre-defined optimization criteria.

In some embodiments of the invention, one or a plurality of measurements of brain activity, physiology, or cognitive function determines the sequence of delivery for a pre-selected set of bioTU waveforms according to a lookup-table or appropriate mathematical or statistical algorithm. In some embodiments of the invention, one or a plurality of parameters that define a bioTU protocol is determined based upon a measurement of brain activity, physiology, or cognitive function in the user according to a lookup-table or appropriate mathematical or statistical algorithm.

Improvement or optimization of a bioTU waveform is accomplished by iterating (or ‘sweeping’) through multiple bioTU waveforms. Embodiments of the present invention incorporate one or more hardware and/or software components and related methods for improving or optimizing a bioTU waveform.

In various embodiments, the optimization criteria includes one or more from the group of: species, individual, health or wellness information about the individual, demographic information about the individual, brain target, transducer location, transducer specifications, intended neuromodulatory effect, or other characteristic relevant to the bioTU session. Appropriate signal processing techniques can improve prediction of how well a bioTU protocol will work by taking into account relevant metadata.

The unrestricted search space of all bioTU waveforms is vast, as it represents all waveforms, and thus can be infinitely varied at each point in time. Realistic limitations can be imposed, coming from safety constraints (e.g. tissue heating, tissue damage threshold), efficacy measurements (e.g. a change in cognitive function or brain activity), technical constraints (e.g. transducer bandwidth, sampling rate of waveform generators), or a combination of more than one of technical, safety, and efficacy constraints (e.g. FDA limits to exposure resulting from biological concerns and/or implementation of device and waveforms).

In some embodiments of the invention, the parameter space to explore is reduced by constraining one or more parameters including, but not limited to, the duration of the entirety of ultrasound pulses during a session, the sampling rate for the generation of the waveform, RF amplifier bandwidth, transducer bandwidth, and amplitude of waveforms.

An example of one reduced waveform space is the space of waveforms that are generated by the layering of component waveforms, where layering includes, but is not limited to, multiplication of waveforms, convolution, deconvolution, triggering at a threshold of a waveform, and the combined triggering and multiplication of waveforms. These component waveforms are generated from pulses that are repeated either in a periodic fashion (giving rise to periodic waveforms), random triggering (for example, where a single 1 μs wide sine pulse is repeated every 1 to 5 μs at a time determined by a random number generator), or by defining a function that governs triggering (chirps, or frequency sweeps, being one such example.) Furthermore, component waveforms, once generated, can undergo additional temporal manipulations. For instance, chirps can also be generated in this fashion, wherein instead of linear time, the waveforms are played back under quadratic or exponential time.

Modulation of parameters such as frequency and the addition of noise to the waveform is an additional manner of transformation or manipulation, whereby one can reduce electrical interference with components of the system used for monitoring brain activity or physiology.

In various embodiments of the invention, an optimization criterion or a plurality of optimization criteria are chosen for use by the algorithm used to select a bioTU waveform to be delivered. In various embodiments of the invention, stored metadata is analyzed using one or a plurality of statistical techniques to select or generate a subsequent bioTU waveform to be delivered to a subject. The choice of the optimization criteria can be made to take advantage of known aspects of transcranial ultrasound transmission and neuromodulation by bioTU.

In some embodiments of the invention, statistical analysis routines are achieved by a component of the bioTU system wearably attached to the user. In some embodiments of the invention, the statistical analysis routines are achieved by a system remote from the bioTU system wearably attached to the user. In some embodiments of the invention, the statistical analysis routines achieved by a system remote from the bioTU system wearably attached to the user are transmitted to the bioTU system wearably attached to the user by a wireless or wired communication protocol. In some embodiments of the invention, the signaling processing components apply one or a plurality of statistical or mathematical algorithms for optimization.

In some embodiments of the invention, the statistical technique includes one or a plurality of automated or supervised normalization routines. In some embodiments of the invention, the statistical technique is used to select one or a plurality of bioTU waveforms to deliver to a subject. In some embodiments of the invention, the at least one statistical technique is chosen from the group of: data mining, machine learning, artificial neural network, artificial intelligence, feature selection, dimensional reduction, feature extraction, principal components analysis, singular value decomposition, multifactor dimensionality reduction, multilinear subspace learning, nonlinear dimensionality reduction, Isomap, kernal principal components analysis, multilinear principal components analysis, Fourier-related transforms, or topological data analysis.

In some embodiments of the invention, the one or a plurality of algorithms for optimization is a form of multi-objective optimization. In some embodiments of the invention, the one or a plurality of algorithms for optimization is a form of iterative optimization. In some embodiments of the invention, the one or a plurality of algorithms for optimization is a form of gradient search optimization. In some embodiments of the invention, the one or a plurality of algorithms for optimization computes a Hessian matrix.

In some embodiments of the invention, the one or a plurality of algorithms for optimization is one or a plurality of heuristic or metaheuristic algorithms. In some embodiments of the invention, the one or a plurality of heuristic or metaheuristic algorithms is a form of genetic algorithm, simulated annealing, tabu search, differential evolution, dynamic relaxation, hill climbing, Nelder-Mead method, or particle swarm optimization.

In some embodiments of the invention, the search algorithms for optimization are written as software or achieved in hardware by a digital circuit design. In some embodiments of the invention, the signaling processing components select the at least one bioTU waveform or bioTU waveform component using random, pseudo-random, or chaotic statistical or mathematical techniques. In some embodiments of the invention, the bioTU waveform is chosen from a list. In some embodiments of the invention, the bioTU waveform is selected manually by a skilled practitioner of bioTU.

An exemplar embodiment of the invention is a device for determining effective bioTU parameters for modulating neural activity. Computerized system 1408 transmits a waveform 1401 to waveform generator 1402 that sends analog information to radiofrequency (RF) amplifier 1403 that drives ultrasound transducer 1404 to deliver ultrasound energy to a subject and induce ultrasound neuromodulation.

In an embodiment, the assessment of the effectiveness of a bioTU protocol is measured with electrodes implanted in a non-human primate. The electrodes are configured to record neural activity of one or many neurons. The recorded signal is filtered and amplified 1405 then transmitted to data acquisition board 1406 where it is digitized 1407 and transmitted to computerized system 1408 for one or more of data analysis 1409, recording of information to a waveform bank 1410, querying of a waveform bank, 1411, and performance of an appropriate statistical analysis to determine a next bioTU waveform 1412.

In an alternative exemplar embodiment, ultrasound transducer configured to deliver bioTU to a subject 1404 is targeted to modulate attentional state of subject 1413 which is assessed by a video-based eye-tracking system that determines a subject's gaze 1414, then digitized 1406 and transmitted to a laptop 1408 for analysis 1409, saving data to a waveform bank 1410, and selection of a next bioTU waveform 1412.

In yet another exemplar embodiment, a smartphone app determines a first bioTU waveform to be delivered and transmits a signal wirelessly by Bluetooth to a headband-mounted ultrasound transducer to trigger delivery of the first bioTU waveform. The subject's response is measured by electroencephalography using an Avatar battery-operated wireless amplifier that transmits a recorded signal to the smartphone. The app processes the received signal and selects a subsequent bioTU waveform to be transmitted to the wearably attached ultrasound stimulation components for delivering a bioTU protocol to the subject.

### DEFINITIONS

As used herein, the terms ‘brain stimulation’, ‘neuromodulation’, and ‘neuronal activation’ interchangeably to refer to invasive or non-invasive techniques to alter the excitability, action potential rate, vesicular release rate, or other biochemical pathway in neurons or other cell types in the brain.

As used herein, the terms “bioTU”, “bioTU protocol”, ‘bioTU stimulation protocol’, ‘bioTU stimulation waveform’, ‘transcranial ultrasound neuromodulation protocol’, ‘ultrasound stimulation protocol’, ‘ultrasound stimulation waveform,” and “bioTU stimulation” interchangeably to refer a modulation of brain circuit activity induced by patterned, local vibration of brain tissue using US whereby:
Ultrasound is transmitted into the brain;
A dominant acoustic frequency is generally greater than about 100 kHz and less than about 10 MHz. Particularly advantageous acoustic frequencies are between about 0.3 MHz and 0.7 MHz;
The spatial-peak temporal-average (Iₛₚₜₐ) intensity of the ultrasound waveform at the brain tissue is less than about 1 W/cm². Particularly advantageous Iₛₚₜₐ values are between about 100 mW/cm² and about 700 mW/cm².
The ultrasound pulse length is less than about 5 seconds; and
The protocol induces an effect in one or more brain regions such as neuromodulation, brain activation, neuronal activation, neuronal inhibition, or a change in blood flow whereby heating of brain tissue does not exceed approximately 2 degrees Celsius for a period greater than about 5 seconds.

As used herein, mechanical effects of ultrasound waves in the brain are defined as effects caused by the local vibration of brain tissue. Thermal effects of ultrasound waves in the brain are defined as effects caused by the heating of brain tissue.

As used herein, the term “pulse length” is defined as the amount of time of a non-interrupted tone burst of one or more ultrasound acoustic wave frequency components.

As used herein, the term “pulse repetition period” is defined to be the amount of time between the onset of consecutive ultrasound pulses. The “pulse repetition frequency” is equivalent to the inverse of the “pulse repetition period”.

As used herein, the term “bioTU waveform” is defined as a period of ultrasound delivered with a pulsed or continuous wave construction or more complex waveform. bioTU waveforms may be that includes a specified number of pulses that may be repeated at the pulse repetition frequency. In some cases, a bioTU waveform is composed of a single continuous wave tone burst of greater than about one second that is not repeated. In such cases, the “pulse length” and “bioTU waveform duration” may be about equal.

As used herein, the term “bioTU waveform component” is defined as a feature of a bioTU waveform that, in isolation, is insufficient to fully define a bioTU waveform.

As used herein, the term “bioTU repetition period” is defined as the amount of time of between the onset of consecutive bioTU waveforms. The “bioTU repetition frequency” is equivalent to the inverse of the “bioTU repetition period”.

As used herein, the terms “waveform bank”, “ultrasound waveform bank”, “bioTU waveform bank”, and “relational database” are used interchangeably to refer to a database (or data store) of one or a plurality of ultrasound waveforms that may optionally also include one or a plurality of ultrasound waveform component. The waveform bank may be stored on electronic media in any form known to one skilled in the art of database design. In some embodiments, the waveform bank is stored in a database system that is a component of a system wearably attached or near to the user. In alternative embodiments, the waveform bank is stored in a database system remote from the user that connects to a bioTU system wearably attached to the user directly by a wireless or wired communication protocol or via a local area network, wide area network (e.g., the Internet). In some embodiments, the waveform bank stores metadata including one or a plurality from the group of bioTU waveform parameters, hardware components for delivering bioTU, software associated with hardware components for delivering bioTU, the intended target, the intended neuromodulatory effect, the intended change to cognitive state, cognitive function, or sensory processing, and metadata about the user's health, genetics, behavior, emotional state, physical characteristics, diet, drug use (approved prescription drugs and illegal drugs), alcohol use, or other characteristic of the user.

As used herein, the term “metadata” refers to information about the bioTU system, bioTU user, intended one or more brain targets, intended one or more neuromodulatory effect, actual one or more neuromodulatory effects, and other information related to a bioTU session.

As used herein, the term “bioTU assessment” refers to one more measurements that assess the safety, efficacy, and/or efficiency of ultrasound transmission to the one or more targeted brain regions.

As used herein, the singular forms “a,” “an,” and “the” include plural referents unless the context clearly dictates otherwise. Thus, for example, reference to “an ultrasound waveform” includes mixtures of two or more ultrasound waveforms, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, reference will be made to a number of terms which shall be defined to have the following meanings:

“Optional” or “optionally” means that the subsequently described event or circumstance may or may not occur, and that the description includes instances where said event or circumstance occurs and instances where it does not. The term “treating” refers to inhibiting, preventing, curing, reversing, attenuating, alleviating, minimizing, suppressing or halting the deleterious effects of a disease and/or causing the reduction, remission, or regression of a disease. Those of skill in the art will understand that various methodologies and assays can be used to assess the development of a disease, and similarly, various methodologies and assays may be used to assess the reduction, remission or regression of the disease.

“Increase” is defined throughout as less than a doubling such as an increase of 5%, 10%, or 50% or as an increase of 1, 2, 3, 4, 5, 6, 7, 8, 9, 10, 11, 12, 13, 14, 15, 16, 17, 18, 19, 20 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 51, 52, 53, 54, 55, 56, 57, 58, 59, 60, 61, 62, 63, 64 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 400, or 500 times increase as compared with basal levels or a control.

What is claimed is:

1. A system for delivering and assessing transcranial ultrasound neuromodulation protocols during a transcranial ultrasound neuromodulation session, said system comprising:
at least one control component configured to select at least one ultrasound waveform to deliver to a subject;
at least one stimulation component configured to deliver said at least one ultrasound waveform to the brain of the subject; and
at least one transcranial ultrasound neuromodulation assessment component configured to measure one or more changes in brain or body of the subject induced by transcranial ultrasound neuromodulation,
wherein said at least one control component, at least one stimulation component, and at least one transcranial ultrasound neuromodulation assessment component are configured to communicate and operate in conjunction with one another in order to:
select a plurality of transcranial ultrasound neuromodulation waveforms, wherein each one of said plurality of transcranial ultrasound neuromodulation waveforms is (i) selectable by said at least one control component, (ii) configured to be delivered to the brain of said subject by said at least one stimulation component, and (iii) capable of generating a response measured for efficacy by at least one of said at least one transcranial ultrasound neuromodulation assessment component.

2. The system as described in claim 1, wherein the one or more changes in the brain or body measured by said at least one transcranial ultrasound neuromodulation assessment component include one or more changes selected from the group consisting of brain activity, physiology, and cognitive function.

3. The system as described in claim 1, further comprising a waveform bank comprising a storage medium configured to receive and store metadata and communicative with one or more of said at least one control component, at least one stimulation component and at least one transcranial ultrasound neuromodulation assessment component, wherein said metadata comprises one or more data components selected from the group consisting of information about the transmitted transcranial ultrasound neuromodulation waveform, information about said at least one control component, information about said at least one stimulation component, information about said at least one transcranial ultrasound neuromodulation assessment component, information about said subject, information about one or more measurements taken by at least one of said at least one transcranial ultrasound neuromodulation assessment component, information about one or more intended brain targets, information about one or more intended neuromodulatory effects, information about one or more actual neuromodulatory effects, and information about one or more transcranial ultrasound neuromodulation sessions.

4. The system as described in claim 3, wherein said metadata stored in said waveform bank is utilized by at least one of said at least one control component in selecting one or more of said plurality of transcranial ultrasound neuromodulation waveforms.

5. The system as described in claim 3, wherein said metadata stored in said waveform bank is updated after delivery of a transcranial ultrasound neuromodulation waveform to said subject.

6. The system as described in claim 3, wherein the stored metadata is analyzed using one or a plurality of statistical techniques.

7. The system as described in claim 1, wherein at least one of said plurality of transcranial ultrasound neuromodulation waveforms comprises a complex transcranial ultrasound neuromodulation waveform, wherein said complex transcranial ultrasound neuromodulation waveform is generated by at least one of said at least one stimulation component and is formed through one or more methods selected from the group comprising adding two or more transcranial ultrasound neuromodulation waveforms, subtracting two or more transcranial ultrasound neuromodulation waveforms, hybridizing two or more transcranial ultrasound neuromodulation waveforms, concatenating two or more transcranial ultrasound neuromodulation waveforms, convolving two or more transcranial ultrasound neuromodulation waveforms, multiplying two or more transcranial ultrasound neuromodulation waveforms, dividing two or more transcranial ultrasound neuromodulation waveforms, combining two or more transcranial ultrasound neuromodulation waveforms through temporal offsets, combining two or more transcranial ultrasound neuromodulation waveforms through voltage offsets, modulating amplitude of one or more transcranial ultrasound neuromodulation waveforms, and combining two or more transcranial ultrasound neuromodulation pulse trains.

8. The system as described in claim 1, wherein said plurality of transcranial ultrasound neuromodulation waveforms target a plurality of brain regions.

9. The system as described in claim 1, wherein each of said plurality of transcranial ultrasound neuromodulation waveforms differ from the other transcranial ultrasound neuromodulation waveforms of said plurality of transcranial ultrasound neuromodulation waveforms in one or more of spatial-peak temporal-average intensity, acoustic frequency, pulse length, pulse repetition frequency, number of pulses, brain region targeted, and stimulation component utilized.

10. The system as described in claim 1, wherein said at least one control component, at least one stimulation component, and said at least one transcranial ultrasound neuromodulation assessment component are wearably attached to the subject.

11. The system as described in claim 5, wherein said metadata is utilized by said at least one of said at least one control component to optimize an assessment made of the subject in response to one or more of said plurality of transcranial ultrasound neuromodulation waveforms.

12. The system as described in claim 11, wherein at least one of said at least one transcranial ultrasound neuromodulation assessment component comprises at least one user interface component configured to allow said subject to report a subjective experience that occurs in response to transcranial ultrasound neuromodulation stimulation that takes the form of one or a plurality of subjective experiences selected from the group consisting of: a sensory perception, movement, concept, instruction, other symbolic communication, or a modification of the recipient's cognitive, emotional, physiological, attentional, and other cognitive state.

13. The system as described in claim 11, further comprising one or a plurality of components for measuring brain activity by a technique selected from the group consisting of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, and other technique for measuring brain activity known to one skilled in the art.

14. The system as described in claim 11, further comprising one or a plurality of components for making a physiological measurement of the body selected from the group consisting of: electromyogram (EMG), galvanic skin response (GSR), heart rate, blood pressure, respiration rate, pulse oximetry, pupil dilation, eye movement, gaze direction, and other physiological measurement known to one skilled in the art.

15. The system as described in claim 11, further comprising one or a plurality of components for making a cognitive assessment selected from the group consisting of: a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, and a customized cognitive task.

16. A method for delivering and assessing transcranial ultrasound neuromodulation protocols during a transcranial ultrasound neuromodulation session, said method comprising the steps of:
selecting a first transcranial ultrasound neuromodulation waveform to deliver to a subject;
delivering said first transcranial ultrasound neuromodulation waveform to said subject with at least one stimulation component;
assessing a first set of one or more changes in brain or body of the subject induced by said first transcranial ultrasound neuromodulation waveform;
selecting a second transcranial ultrasound neuromodulation waveform to deliver to said subject, wherein said second transcranial ultrasound neuromodulation waveform is different in one or more characteristics from said first transcranial ultrasound neuromodulation waveform;
delivering said second transcranial ultrasound neuromodulation waveform to said subject with said at least one stimulation component; and
assessing a second set of one or more changes in brain or body of the subject induced by said second transcranial ultrasound neuromodulation waveform.

17. The method as described in claim 16, further comprising the steps of:
receiving metadata at a waveform bank comprising a storage medium and communicative with one or more of said at least one control component, at least one stimulation component, and at least one transcranial ultrasound neuromodulation assessment component, wherein said metadata comprises one or more data components selected from the group consisting of information about the transmitted transcranial ultrasound neuromodulation waveform, information about said at least one control component, information about said at least one stimulation component, information about said at least one transcranial ultrasound neuromodulation assessment component, information about said subject, information about one or more measurements taken by at least one of said at least one transcranial ultrasound neuromodulation assessment component, information about one or more intended brain targets, information about one or more intended neuromodulatory effects, information about one or more actual neuromodulatory effects, and information about one or more transcranial ultrasound neuromodulation sessions; and
storing said metadata in said waveform bank.

18. The method as described in claim 16, wherein said metadata stored in said waveform bank is utilized by said at least one control component in selecting said second transcranial ultrasound neuromodulation waveform and wherein said waveform bank comprises a computer readable medium.

19. The method as described in claim 18, further comprising the step of updating said metadata stored in said waveform bank after delivery of said first transcranial ultrasound neuromodulation waveform to said subject.

20. The method as described in claim 16, wherein at least one of said first transcranial ultrasound neuromodulation waveform and said second transcranial ultrasound neuromodulation waveform comprises a complex transcranial ultrasound neuromodulation waveform, wherein said complex transcranial ultrasound neuromodulation waveform is generated by at least one of said at least one stimulation component and is formed through one or more methods selected from the group comprising adding two or more transcranial ultrasound neuromodulation waveforms, subtracting two or more transcranial ultrasound neuromodulation waveforms, hybridizing two or more transcranial ultrasound neuromodulation waveforms, concatenating two or more transcranial ultrasound neuromodulation waveforms, convolving two or more transcranial ultrasound neuromodulation waveforms, multiplying two or more transcranial ultrasound neuromodulation waveforms, dividing two or more transcranial ultrasound neuromodulation waveforms, combining two or more transcranial ultrasound neuromodulation waveforms through temporal offsets, combining two or more transcranial ultrasound neuromodulation waveforms through voltage offsets, modulating amplitude of one or more transcranial ultrasound neuromodulation waveforms, and combining two or more transcranial ultrasound neuromodulation pulse trains.

21. The method as described in claim 16, wherein said first and second transcranial ultrasound neuromodulation waveforms differ from the other in one or more of spatial-peak temporal-average intensity, acoustic frequency, pulse length, pulse repetition frequency, number of pulses, brain region targeted, and stimulation component utilized.

22. The method as described in claim 18, further comprising the step of:
optimizing characteristics of one or more of said first and second transcranial ultrasound neuromodulation waveforms based on said metadata.

23. The method as described in claim 16, further comprising the step of making an assessment of a response in a subject in response to said first and second transcranial ultrasound neuromodulation waveforms.

24. The method as described in claim 23, wherein the assessment is achieved by prompting a subject to report by using one or a plurality of user interface components a subjective experience that occurs in response to transcranial ultrasound neuromodulation stimulation selected from the group consisting of: a sensory perception, movement, concept, instruction, other symbolic communication, or a modification of the recipient's cognitive, emotional, physiological, attentional, and other cognitive state.

25. The method as described in claim 23, wherein the assessment is achieved by measuring brain activity by a technique selected from the group consisting of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, and other techniques for measuring brain activity known to one skilled in the art.

26. The method as described in claim 23, wherein the assessment is achieved by making a physiological measurement of the body selected from the group consisting of: electromyogram (EMG), galvanic skin response (GSR), heart rate, blood pressure, respiration rate, pulse oximetry, pupil dilation, eye movement, gaze direction, and other physiological measurement known to one skilled in the art.

27. The method as described in claim 23, wherein the assessment is achieved by making a cognitive assessment selected from the group consisting of: a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, and a customized cognitive task.

28. A system for treating a subject with transcranial ultrasound neuromodulation, said system comprising:
at least one control component configured to select at least one ultrasound waveform to deliver to a subject;
at least one stimulation component configured to deliver said at least one ultrasound waveform to the brain of the subject; and
at least one transcranial ultrasound neuromodulation assessment component configured to measure one or more changes in the brain or body of the subject induced by therapeutic ultrasound;
wherein said at least one control component or said at least one transcranial ultrasound neuromodulation assessment component comprises a computer readable memory having instructions of a computer program to identify a transcranial ultrasound neuromodulation waveform among a plurality of transcranial ultrasound neuromodulation waveforms in response to measured efficacy of said transcranial ultrasound neuromodulation waveform.

29. A method of treating a subject with ultrasound, said method comprising the steps of:
delivering a plurality of transcranial ultrasound neuromodulation waveforms to a subject with at least one stimulation component;
assessing a response of the subject to each of the plurality of transcranial ultrasound neuromodulation waveforms;
identifying a transcranial ultrasound neuromodulation waveform among the plurality of transcranial ultrasound neuromodulation waveforms in response to one or more changes in brain or body of the subject induced by the transcranial ultrasound neuromodulation waveform.
